(19) United States
(12) Patent Application Publication
(10) Pub. No.: US 2024/0203555 A1
(43) Pub. Date: Jun. 20, 2024

(54) METHODS AND SYSTEMS FOR THERAPY MONITORING AND TRIAL DESIGN

(71) Applicant: Scipher Medicine Corporation, Waltham, MA (US)

(72) Inventors: Susan Ghiassian, Boston, MA (US); Viatcheslav R. AKMAEV, Sudbury, MA (US); Ivan VOITALOV, Brighton, MA (US)

(21) Appl. No.: 18/544,214

(22) Filed: Dec. 18, 2023

Related U.S. Application Data
(63) Continuation of application No. PCT/US2022/034375, filed on Jun. 21, 2022.
(60) Provisional application No. 63/329,008, filed on Apr. 8, 2022.
(60) Provisional application No. 63/213,431, filed on Jun. 22, 2021.

(51) Int. Cl. G16H 20/10; A61K 45/06; C07K 16/24; G16B 20/20; A61K 39/00

(52) U.S. Cl. CPC G16H 20/10; A61K 45/06; C07K 16/241; G16B 20/20; A61K 2039/505

(57) ABSTRACT

Described are methods and systems for identifying a disease gene expression signature determined to revert a disease gene expression signature in a subject suffering from a disease to a non-diseased expression signature (e.g., gene expression of a non-diseased subject). Also provided herein are methods of designing a study (e.g., a clinical trial) comprising identifying diseased subjects who exhibit a quantifiable change in the disease gene expression signature towards gene expression of a non-diseased subject.

### CROSS-REFERENCE TO RELATED APPLICATIONS

This application is a continuation of International Application No. PCT/US2022/034375, filed Jun. 21, 2022, which claims priority to U.S. Provisional Application No. 63/213,431, filed Jun. 22, 2021, and U.S. Provisional Application No. 63/329,008, filed Apr. 8, 2022, each of which is incorporated by reference herein in its entirety.

### BACKGROUND

Therapy response for many complex diseases may continue to elude researchers and practitioners. A single stratification factor or biomarker may be insufficient to determine whether a therapy is effective in treating a particular patient. Instead, many diseases, such as autoimmune diseases, cancers, and the like, affect a multitude of biological sub-systems. (See e.g., Frohlich, et al., BMC Med, 16, 150:1122-1127 (2018), which is incorporated herein by reference for all purposes). A reactive approach (e.g., a trial-and-error approach) to identifying treatment for patients may be costly and introduce risk for adverse side effects, potential disease progression, and delay of proper treatment. (See e.g., Mathur & Sutton, Biomed. Rep., 7:3-5 (2017), which is incorporated herein by reference for all purposes).

Further, inability to determine individual response vs. therapy efficacy in clinical trials may be costly and dangerous for subjects who are only exposed to dangerous side effects without any benefit from the therapy. Once a subject is determined to be a non-responder to a particular therapy on an individual basis, they can be removed from a clinical trial, but such a delay in removal may increase the risk of serious side effects, as well as lost time and cost for the study directors. Predicting which subjects may be responsive or non-responsive early in a clinical trial may be challenging or may not always be possible if there are no predictive biomarkers available. Changes in clinical characteristics, instead, may be used determine whether a subject is or is not responding to a therapy, but such changes can take time, and be subjective in nature, especially for changes that are marked by self-assessment of individual subjects.

### SUMMARY

To date, many approaches to determining suitability of a therapy for a particular subject may rely on a reactive approach of attempting multiple therapies, attempting to gauge patient response by assessing clinical characteristics. These approaches may delay necessary treatment and may mischaracterize the actual responsiveness of a therapy for a patient by only examining clinical characteristics of response. Therefore, there is a need for methods and systems of providing personalized treatments for patients that reliably quantify responsiveness to therapy.

The present disclosure provides methods and systems that encompass an insight that treating a patient on a molecular level, e.g., providing a treatment that converts a subset of a gene expression profile from a diseased subject to resemble the gene expression profile a healthy subject, proactively, may be a better metric for assessing drug molecular response and identifying effective therapy than by a reactive approach, or seeking out a singly one-size-fits-all biomarker. Provided technologies, among other things, permit providers to identify particular methods and modes of treatment that may work for that particular patient and allow providers to monitor disease progression and treatment response without relying on subjective measures, such as clinical characteristics or patient self-assessment. In some embodiments, changes in certain gene expression patterns for diseased patients are indicative of a response to therapy, and reversal of gene expression of this gene expression pattern in a diseased patient indicates improvement of the health of the diseased subject (“a disease gene expression signature”). Such an approach is distinct from other methods, which compare gene expression differences between patients suffering from the disease (e.g., an intra-cohort examination), in order to identify whether a patient has a biomarker or expression profile indicative for response to therapy, as compared to other patients who do not.

In some embodiments, reversal of gene expression of some or all of the genes in a disease gene expression signature may cause a diseased subject's gene expression to resemble that of a healthy control subject. Reversal of some or all (e.g., all or substantially all) of gene expression for genes within a disease gene expression signature may indicate regression of the disease, and that the subject may return to a healthy state. In some embodiments, reversal of a disease gene expression signature is achieved by a therapy that modulates one or more genes of the disease gene expression signature.

In some embodiments, a disease gene expression signature is identified using a machine learning algorithm that identifies genes that are differentially expressed between diseased subjects, subsets of diseased subjects, and healthy subjects in a significant manner. Moreover, the present disclosure provides methods and systems that encompass an insight that certain genes within a gene expression profile of a disease subject, when compared to the gene expression profile of a healthy subject, lead to potential targets for therapy that are distinct from the differentially expressed genes in the diseased subject as compared to the healthy subject. That is, while other methods focus on differentially expressed genes in a diseased subject vs. a healthy subject, methods and systems of the present disclosure instead may identify targets for therapy that have significant connection (and thus impact) to these differentially expressed genes but may not be differentially expressed themselves as between diseased and healthy subjects.

In some embodiments, the present disclosure provides methods and systems that encompass an insight that subjects suffering from a disease can be stratified as responders or non-responders to particular therapies by analysis of changes in gene expression in a disease gene expression signature after administration of therapy. Such a change may be observable sooner than changes in clinical characteristics that may be used to determine responsiveness to therapy, (e.g., a non-responder could cease therapy or be removed from a clinical trial before too much time and cost has been lost).

In an aspect, the present disclosure provides a method of determining a disease gene expression signature for quantifying responsiveness to a therapy for subjects suffering from a disease, disorder, or condition, the method comprising: receiving gene expression data from a cohort of subjects suffering from the same disease, disorder, or condition; stratifying the cohort of subjects into two or more groups based at least in part on the gene expression data; calculating differences in gene expression between the two or more groups of subjects and a group of non-diseased subjects; selecting one or more genes having significant differences in gene expression between the two or more groups of subjects and the group of non-diseased subjects (“disease candidate genes”); compiling a set of disease genes comprising the disease candidate genes; and selecting at least a subset of the set of disease genes to thereby determine the disease gene expression signature.

In some embodiments, the method further comprises mapping the disease candidate genes onto a biological network, and selecting adjacent genes on the biological network having significant connection to each other or to the disease candidate genes, wherein the set of disease genes comprises the disease candidate genes and the adjacent genes. In some embodiments, the biological network comprises a human interactome. In some embodiments, the adjacent genes form a significant sub-network with each other or to the disease candidate genes. In some embodiments, the adjacent genes are identified via a machine-learning algorithm. In some embodiments, the machine-learning algorithm comprises a random walk.

In some embodiments, the disease, disorder, or condition comprises ulcerative colitis, Crohn's disease, rheumatoid arthritis, juvenile arthritis, psoriatic arthritis, plaque psoriasis, ankylosing spondylitis, Guillain-Barre syndrome, Sjogren's syndrome, scleroderma, vitiligo, bipolar disorder, Graves' disease, schizophrenia, Alzheimer's disease, multiple sclerosis, Parkinson's disease, or a combination thereof. In some embodiments, the disease, disorder, or condition comprises ulcerative colitis. In some embodiments, the disease, disorder, or condition comprises rheumatoid arthritis. In some embodiments, the disease, disorder, or condition comprises Alzheimer's disease. In some embodiments, the disease, disorder, or condition comprises multiple sclerosis.

In some embodiments, stratifying the cohort of subjects into two or more groups is random or based at least in part on whether the prior subjects do or do not respond to the therapy. In some embodiments, the therapy comprises a member selected from Table 1. In some embodiments, the therapy comprises an anti-TNF therapy. In some embodiments, the cohort of subjects is suffering from the same disease, disorder, or condition as the subjects being assessed for therapy responsiveness. In some embodiments, the stratifying further comprises grouping subjects from the same cohort having similar gene expression.

In some embodiments, the method further comprises using the disease gene expression signature to train a machine learning classifier, wherein the trained machine learning classifier is configured to predict responsiveness or non-responsiveness of a test subject suffering from the disease, disorder, or condition to the therapy, based at least in part on analyzing gene expression data of the test subject.

In some embodiments, the trained machine learning classifier is configured to predict responsiveness or non-responsiveness of the test subject with an accuracy of at least about 50%, 55%, 60%, 65%, 70%, 75%, 80%, 85%, 90%, or 95%. In some embodiments, the trained machine learning classifier is configured to predict responsiveness or non-responsiveness of the test subject with a sensitivity of at least about 50%, 55%, 60%, 65%, 70%, 75%, 80%, 85%, 90%, or 95%. In some embodiments, the trained machine learning classifier is configured to predict responsiveness or non-responsiveness of the test subject with a specificity of at least about 50%, 55%, 60%, 65%, 70%, 75%, 80%, 85%, 90%, or 95%. In some embodiments, the trained machine learning classifier is configured to predict responsiveness or non-responsiveness of the test subject with a positive predictive value of at least about 50%, 55%, 60%, 65%, 70%, 75%, 80%, 85%, 90%, or 95%. In some embodiments, the trained machine learning classifier is configured to predict responsiveness or non-responsiveness of the test subject with a negative predictive value of at least about 50%, 55%, 60%, 65%, 70%, 75%, 80%, 85%, 90%, or 95%. In some embodiments, the trained machine learning classifier is configured to predict responsiveness or non-responsiveness of the test subject with a true positive rate of at least about 50%, 55%, 60%, 65%, 70%, 75%, 80%, 85%, 90%, or 95%. In some embodiments, the trained machine learning classifier is configured to predict responsiveness or non-responsiveness of the test subject with a true negative rate of at least about 50%, 55%, 60%, 65%, 70%, 75%, 80%, 85%, 90%, or 95%. In some embodiments, the trained machine learning classifier is configured to predict responsiveness or non-responsiveness of the test subject with an area under curve (AUC) of at least about 50%, 55%, 60%, 65%, 70%, 75%, 80%, 85%, 90%, or 95%.

In some embodiments, the method further comprises administering to the test subject a therapeutically effective amount of the therapy, when the trained machine learning classifier predicts responsiveness of the test subject to the therapy. In some embodiments, the method further comprises administering to the test subject a therapeutically effective amount of a second therapy that is different from the therapy, when the trained machine learning classifier predicts non-responsiveness of the test subject to the therapy.

In another aspect, the present disclosure provides a method comprising administering to a test subject a therapeutically effective amount of (i) a therapy, based at least in part on a trained machine learning classifier analyzing a disease gene expression signature to predict responsiveness of the test subject to the therapy, or (ii) a second therapy different from the therapy, based at least in part on the trained machine learning classifier analyzing the disease gene expression signature to predict non-responsiveness of the test subject to the therapy, wherein the disease gene expression signature is determined at least in part by: receiving gene expression data from a cohort of subjects suffering from the disease, disorder, or condition; stratifying the cohort of subjects into two or more groups based at least in part on the gene expression data; calculating differences in gene expression between the two or more groups of subjects and a group of non-diseased subjects; selecting one or more genes having significant differences in gene expression between the two or more groups of subjects and the group of non-diseased subjects (“disease candidate genes”); compiling a set of disease genes comprising the disease candidate genes; and selecting at least a subset of the set of disease genes to thereby determine the disease gene expression signature.

In some embodiments, the disease gene expression signature is determined at least in part by further mapping the disease candidate genes onto a biological network, and selecting adjacent genes on the biological network having significant connection to each other or to the disease candidate genes, wherein the set of disease genes comprises the disease candidate genes and the adjacent genes. In some embodiments, the biological network comprises a human interactome. In some embodiments, the adjacent genes form a significant sub-network with each other or to the disease candidate genes. In some embodiments, the adjacent genes are identified via a machine-learning algorithm. In some embodiments, the machine-learning algorithm comprises a random walk.

In some embodiments, the disease, disorder, or condition comprises ulcerative colitis, Crohn's disease, rheumatoid arthritis, juvenile arthritis, psoriatic arthritis, plaque psoriasis, ankylosing spondylitis, Guillain-Barre syndrome, Sjogren's syndrome, scleroderma, vitiligo, bipolar disorder, Graves' disease, schizophrenia, Alzheimer's disease, multiple sclerosis, Parkinson's disease, or a combination thereof. In some embodiments, the disease, disorder, or condition comprises ulcerative colitis. In some embodiments, the disease, disorder, or condition comprises rheumatoid arthritis. In some embodiments, the disease, disorder, or condition comprises Alzheimer's disease. In some embodiments, the disease, disorder, or condition comprises multiple sclerosis.

In some embodiments, stratifying the cohort of subjects into two or more groups is random or based at least in part on whether the prior subjects do or do not respond to the therapy. In some embodiments, the therapy comprises a member selected from Table 1. In some embodiments, the therapy comprises an anti-TNF therapy. In some embodiments, the stratifying further comprises grouping subjects from the same cohort having similar gene expression.

In some embodiments, the trained machine learning classifier is configured to predict responsiveness or non-responsiveness of the test subject with an accuracy of at least about 50%, 55%, 60%, 65%, 70%, 75%, 80%, 85%, 90%, or 95%. In some embodiments, the trained machine learning classifier is configured to predict responsiveness or non-responsiveness of the test subject with a sensitivity of at least about 50%, 55%, 60%, 65%, 70%, 75%, 80%, 85%, 90%, or 95%. In some embodiments, the trained machine learning classifier is configured to predict responsiveness or non-responsiveness of the test subject with a specificity of at least about 50%, 55%, 60%, 65%, 70%, 75%, 80%, 85%, 90%, or 95%. In some embodiments, the trained machine learning classifier is configured to predict responsiveness or non-responsiveness of the test subject with a positive predictive value of at least about 50%, 55%, 60%, 65%, 70%, 75%, 80%, 85%, 90%, or 95%. In some embodiments, the trained machine learning classifier is configured to predict responsiveness or non-responsiveness of the test subject with a negative predictive value of at least about 50%, 55%, 60%, 65%, 70%, 75%, 80%, 85%, 90%, or 95%. In some embodiments, the trained machine learning classifier is configured to predict responsiveness or non-responsiveness of the test subject with a true positive rate of at least about 50%, 55%, 60%, 65%, 70%, 75%, 80%, 85%, 90%, or 95%. In some embodiments, the trained machine learning classifier is configured to predict responsiveness or non-responsiveness of the test subject with a true negative rate of at least about 50%, 55%, 60%, 65%, 70%, 75%, 80%, 85%, 90%, or 95%. In some embodiments, the trained machine learning classifier is configured to predict responsiveness or non-responsiveness of the test subject with an area under curve (AUC) of at least about 50%, 55%, 60%, 65%, 70%, 75%, 80%, 85%, 90%, or 95%.

In another aspect, the present disclosure provides a method of validating response to a therapy for a subject suffering from a disease, disorder, or condition, the method comprising: analyzing changes in a disease gene expression signature in the subject after administration of the therapy, wherein the disease gene expression signature is determined to quantify responsiveness to the therapy.

In some embodiments, the disease gene expression signature is determined at least in part by: receiving gene expression data from a cohort of subjects suffering from the disease, disorder, or condition; stratifying the cohort of subjects into two or more groups based at least in part on the gene expression data; calculating differences in gene expression between the two or more groups of subjects and a group of non-diseased subjects; selecting one or more genes having significant differences in gene expression between the two or more groups of subjects and the group of non-diseased subjects (“disease candidate genes”); compiling a set of disease genes comprising the disease candidate genes; and selecting at least a subset of the set of disease genes to thereby determine the disease gene expression signature.

In another aspect, the present disclosure provides a method of monitoring therapeutic efficacy in a subject suffering from a disease, disorder, or condition, the method comprising monitoring changes in a disease gene expression signature after administration of a therapy, wherein the disease gene expression signature has been determined at least in part by: analyzing gene expression data from a cohort of subjects suffering from the same disease, disorder, or condition as the subject; stratifying the cohort of subjects into two or more groups based on the gene expression data; determining differences in gene expression between the two or more groups of subjects and a group of non-diseased subjects; selecting one or more genes having significant differences in gene expression between the two or more groups of subjects and the group of non-diseased subjects (“disease candidate genes”); compiling a set of disease genes comprising the disease candidate genes; and selecting at least a subset of the set of disease genes to thereby determine the disease gene expression signature.

In some embodiments, the disease gene expression signature is determined at least in part by further mapping the disease candidate genes onto a biological network, and selecting adjacent genes on the biological network having significant connection to each other or to the disease candidate genes, wherein the set of disease genes comprises the disease candidate genes and the adjacent genes. In some embodiments, the biological network comprises a human interactome. In some embodiments, the adjacent genes form a significant sub-network with each other or to the disease candidate genes. In some embodiments, the adjacent genes are selected by a machine-learning process.

In some embodiments, the disease, disorder, or condition comprises ulcerative colitis, Crohn's disease, rheumatoid arthritis, juvenile arthritis, psoriatic arthritis, plaque psoriasis, ankylosing spondylitis, Guillain-Barre syndrome, Sjogren's syndrome, scleroderma, vitiligo, bipolar disorder, Graves' disease, schizophrenia, Alzheimer's disease, multiple sclerosis, Parkinson's disease, or a combination thereof. In some embodiments, the disease, disorder, or condition comprises ulcerative colitis. In some embodiments, the disease, disorder, or condition comprises rheumatoid arthritis. In some embodiments, the disease, disorder, or condition comprises Alzheimer's disease. In some embodiments, the disease, disorder, or condition comprises multiple sclerosis.

In some embodiments, stratifying the cohort of subjects into two or more groups is random or based at least in part on whether the prior subjects do or do not respond to the therapy. In some embodiments, the therapy comprises a member selected from Table 1. In some embodiments, the therapy comprises an anti-TNF therapy. In some embodiments, the stratifying further comprises grouping subjects from the same cohort having similar gene expression.

In some embodiments, the method further comprises selecting the test subject for a clinical trial, based at least in part on whether the disease gene expression signature of the test subject exhibits a quantifiable change toward a disease gene expression signature of a non-diseased subject.

In another aspect, the present disclosure provides a method of identifying and selecting subjects for a clinical trial comprising: receiving gene expression data of a cohort of subjects; analyzing the gene expression data to detect the presence of a disease gene expression signature; administering at least one dose of a therapy to the cohort of subjects; identifying changes in the disease gene expression signature relative to gene expression of a non-diseased subject; and selecting subjects for the clinical trial who exhibit a quantifiable change in the disease gene expression signature towards gene expression of a healthy subject, wherein the disease gene expression signature is determined by any of the methods provided herein.

In another aspect, the present disclosure provides a system comprising: a processor of a computing device; and a memory having instructions stored thereon, wherein the instructions, when executed by the processor cause the processor to perform any of the methods provided herein.

In another aspect, the present disclosure provides a method of determining a disease gene expression signature for quantifying responsiveness to a therapy for subjects suffering from a disease, disorder, or condition, the method comprising: receiving gene expression data from a cohort of subjects suffering from the same disease, disorder, or condition (e.g., suffering from the same disease, disorder, or condition as the subjects being assessed for therapy responsiveness); stratifying the cohort of subjects into two or more groups based on the gene expression data (e.g., grouping subjects from the cohort having similar gene expression); calculating differences in gene expression between the two or more groups of subjects and a group of healthy subjects; selecting one or more genes having significant differences in gene expression between the two or more groups of subjects and the group of healthy subjects (“disease candidate genes”); mapping the disease candidate genes onto a biological network (e.g., a human interactome); selecting adjacent genes (e.g., genes on adjacent nodes, for example, on a human interactome map) having significant connection to each other (e.g., forming a significant subnetwork) or to the disease candidate genes; compiling a list of disease genes comprising the disease candidate genes and adjacent genes; selecting some or all of the genes from the list of disease genes to thereby provide the disease gene expression signature.

In some embodiments, the adjacent genes are identified via a machine-learning algorithm.

In some embodiments, the machine-learning process comprises a random walk.

In some embodiments, the disease, disorder, or condition comprises ulcerative colitis, Crohn's disease, rheumatoid arthritis, juvenile arthritis, psoriatic arthritis, plaque psoriasis, ankylosing spondylitis, Guillain-Barre syndrome, Sjogren's syndrome, scleroderma, vitiligo, bipolar disorder, Graves' disease, schizophrenia, Alzheimer's disease, multiple sclerosis, Parkinson's disease, or a combination thereof.

In some embodiments, stratifying the cohort of subjects into two or more groups is random or based at least in part on whether the prior subjects do or do not respond to the therapy.

In some embodiments, the therapy comprises a member selected from Table 1.

In some embodiments, the therapy comprises an anti-TNF therapy.

In another aspect, the present disclosure provides a method of validating response to a therapy for a subject suffering from a disease, disorder, or condition, the method comprising: analyzing changes in a disease gene expression signature in the subject after administration of the therapy, wherein the disease gene expression signature is determined to quantify responsiveness to the therapy.

In some embodiments, the disease gene expression signature is derived by: receiving gene expression data from a cohort of subjects suffering from the same disease, disorder, or condition as the subject; stratifying the cohort of subjects into two or more groups based on the gene expression data (e.g., grouping subjects from the cohort having similar gene expression into a group); calculating differences in gene expression between the two or more groups of subjects and a group of healthy subjects; selecting one or more genes having significant differences in gene expression between the two or more groups of subjects and the group of healthy subjects (“disease candidate genes”); mapping the disease candidate genes onto a biological network (e.g, a human interactome); selecting adjacent genes (e.g., genes on adjacent nodes, for example, on a human interactome map) having significant connection to the disease candidate genes; compiling a list of disease genes comprising the disease candidate genes and adjacent genes; selecting some or all of the genes from the list of disease genes to thereby provide the disease gene expression signature.

In another aspect, the present disclosure provides a method of monitoring therapeutic efficacy in a subject suffering from a disease, disorder, or condition, the method comprising monitoring changes in a disease gene expression signature after administration of a therapy, wherein the disease gene expression signature has been derived by a process comprising: analyzing gene expression data from a cohort of subjects suffering from the same disease, disorder, or condition as the subject, stratifying the cohort of subjects into two or more groups based on the gene expression data (e.g., grouping subjects from the cohort having similar gene expression into a group); determining differences in gene expression between the two or more groups of subjects and a group of healthy subjects; selecting one or more genes having significant differences in gene expression between the two or more groups of subjects and the group of healthy subjects (“disease candidate genes”); mapping the disease candidate genes onto a biological network (e.g., a human interactome); selecting adjacent genes (e.g., genes on adjacent nodes, for example, on a human interactome map) having significant connection to the disease candidate genes; compiling a list of disease genes comprising the disease candidate genes and adjacent genes; selecting some or all of the genes from the list of disease genes to thereby provide the disease gene expression signature.

In some embodiments, the adjacent genes are selected by a machine-learning algorithm.

In some embodiments, the disease, disorder, or condition comprises ulcerative colitis, Crohn's disease, rheumatoid arthritis, juvenile arthritis, psoriatic arthritis, plaque psoriasis, ankylosing spondylitis, Guillain-Barre syndrome, Sjogren's syndrome, scleroderma, vitiligo, bipolar disorder, Graves' disease, schizophrenia, Alzheimer's disease, multiple sclerosis, Parkinson's disease, or a combination thereof.

In some embodiments, stratifying the cohort of subjects into two or more groups is random or based at least in part on whether the prior subjects do or do not respond to the therapy.

In some embodiments, the therapy comprises a member selected from Table 1.

In some embodiments, the therapy comprises an anti-TNF therapy.

In another aspect, the present disclosure provides a method of identifying and selecting subjects for a clinical trial comprising: receiving gene expression data of a cohort subjects; analyzing the gene expression data to detect the presence of a disease gene expression signature; administering at least one dose of a therapy to the subjects; identifying changes in the disease gene expression signature relative to gene expression of a healthy subject; and selecting subjects for the clinical trial who exhibit a quantifiable change in the disease gene expression signature towards gene expression of a healthy subject, wherein the disease gene expression signature is determined by a method described herein.

In another aspect, the present disclosure provides a system for determining or validating responsiveness to therapy for a subject suffering from a disease, the system comprising: a processor of a computing device; and a memory having instructions stored thereon, wherein the instructions, when executed by the processor cause the processor to perform operations of any method described herein.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede or take precedence over any such contradictory material.

### BRIEF DESCRIPTION OF THE DRAWINGS

FIG. 1 depicts an example workflow for identifying a disease expression signature.

FIG. 2 depicts a plot illustrating a 2D representation of gene expression profile of responders and non-responders to treatment at baseline and after treatment as well as healthy controls.

FIGS. 3A-3B are a series of overlapping graphs illustrating that non-responder biomarker set is almost fully contained within responders' biomarker set and responder biomarker set was generally twice lager than non-responder biomarker set for each study cohort (FIG. 3A represents Study 1 of Example 1; FIG. 3B represents Study 2 of Example 1).

FIG. 4 depicts an example network environment and computing devices for use in various embodiments.

FIG. 5 depicts an example of a computing device 500 and a mobile computing device 550 that can be used to implement various techniques provided herein.

FIG. 6 depicts a plot illustrating up and downregulated nodes in response to anti TNF treatment, as clustered and connected on a biological network (e.g., a human interactome map).

FIGS. 7A-7E depicts an overview of the module triad framework. FIG. 7A: The pipeline for discovery of the UC module triad on the Human Interactome: the Response module is derived from differentially expressed genes before and after treatment in the patients with active UC who responded to TNFi therapies (infliximab and golimumab); the Genotype module is derived by mapping the genes associated with UC on the Human Interactome; the Treatment module is derived by selecting the small molecule compounds resulting in the alteration of gene expression of the Response module genes using experimental data in the HT29 cell line and mapping the compounds to their protein targets. Target prioritization based on the discovered module triad: FIG. 7B, FIG. 7D: topological relevance of a node to the Genotype module is measured by computing the average shortest path length of the node to all Genotype module nodes, and comparing it to the empirical distribution of average shortest path lengths to the randomized connected subnetworks of the same size as the Genotype module using Z-score (proximity); FIG. 7C, FIG. 7E: functional similarity of a node to the Treatment module is measured by computing the average diffusion state distance (DSD) of the node to all Treatment module nodes, and comparing it to the empirical distribution of average DSDs to the randomized connected subnetworks of the same size as the Treatment module using Z-score (selectivity). All nodes are ranked based on proximity and selectivity, and their ranks are combined using rank product to obtain the final target ranking.

FIGS. 8A-8B depicts gene expression profiles of normal tissue controls and UC active patients before and after TNFi therapy. The first two coordinates of the UMAP embedding of gene expression profiles are based on the set of 545 differentially expressed genes between patients with active UC and normal controls for: FIG. 8A infliximab TNFi treatment; FIG. 8B golimumab TNFi treatment.

FIGS. 9A-9D depicts recovery of the targets approved for 4 complex diseases based on diffusion state distance (DSD). Receiver operator characteristic (ROC) curves for recovery of know approved targets for treatment of: FIG. 9A Alzheimer's disease; FIG. 9B ulcerative colitis; FIG. 9C rheumatoid arthritis; FIG. 9D multiple sclerosis. Individual ROC curves demonstrate recovery of the approved targets given one know approved target and DSD from it to the rest of the HI nodes. Red lines represent mean ROC curves obtained by averaging over the individual ROC curves, and area under the curve (AUC) is reported for the mean ROC curve.

FIGS. 10A-10C depicts in silico validation of the module triad target prioritization. FIG. 10A: Selectivity-proximity scatter plot of the HI nodes with 23 targets approved for UC treatment highlighted. More selective and proximal targets are located towards the lower left of the scatter plot. FIG. 10B: Receiver operator characteristic (ROC) curves for recovery of the approved UC targets using proximity to the Genotype module, selectivity to the Treatment module, a combination of both, and the Local radiality with respect to the Response module, with corresponding areas under the curve (AUC). FIG. 10C: Violin plots of the combined selectivity-proximity ranks of the targets launched for UC, and targets being at preclinical and clinical trials development stage for UC.

FIGS. 11A-11C depicts an overview of the DE analyses. FIG. 11A: schematic illustration of the differential expression gene sets obtained by comparing different pairs of states of responders, non-responders, and normal controls, with the DE genes set names used throughout the paper specified; FIG. 11B: Venn diagrams for R, NR, and RBA sets in infliximab and golimumab studies; FIG. 11C: mutual overlaps of R, NR, and RBA sets across the studies.

FIGS. 12A-12C depicts a KEGG pathway enrichment analysis for genes differentially expressed in responders and non-responders at the baseline with respect to healthy controls. FIG. 12A: Venn diagram (top) for responders' (R) and non-responders' (NR) differentially expressed genes at the baseline with respect to healthy controls after merging the infliximab—and golimumab-based cohorts. FIG. 12A: Venn diagrams (bottom) for the same gene sets within the KEGG pathways database. FIGS. 12B-12C: KEGG pathways significantly enriched with NR gene set that also have significantly more NR-exclusive genes than R-exclusive genes.

FIG. 13 depicts a number of targets per drug. The majority of drugs approved or being developed for UC treatment have maximum of 4 simultaneous targets. We filter out the drugs with >4 targets in our analysis.

FIG. 14 shows a computer system 1401 that is programmed or otherwise configured to perform analysis or operations of various methods.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided herein are systems and methods that are useful, for example, for determining and validating response to therapy. In some embodiments, the present disclosure provides systems and methods for identifying a set of genes that, when differentially expressed as compared to a healthy subject, indicate response to therapy. In some embodiments, the present disclosure provides systems and methods for patient stratification (e.g., in clinical trials) to identify responders and non-responders to therapy on a molecular level, without needing to rely on changes in clinical characteristics.

### Definitions

Administration: As used herein, the term “administration” generally refers to the administration of a composition to a subject or system, for example to achieve delivery of an agent that is, or is included in or otherwise delivered by, the composition.

Agent: As used herein, the term “agent” generally refers to an entity (e.g., for example, a lipid, metal, nucleic acid, polypeptide, polysaccharide, small molecule, etc., or complex, combination, mixture or system [e.g., cell, tissue, organism] thereof), or phenomenon (e.g., heat, electric current or field, magnetic force or field, etc.).

Amino acid: As used herein, the term “amino acid” generally refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. As used herein, the term “standard amino acid” refers to any of the twenty L-amino acids commonly found in naturally occurring peptides. “Nonstandard amino acid” refers to any amino acid, other than the standard amino acids, regardless of whether it is or can be found in a natural source. In some embodiments, an amino acid, including a carboxy- or amino-terminal amino acid in a polypeptide, can contain a structural modification as compared to the general structure above. For example, in some embodiments, an amino acid may be modified by methylation, amidation, acetylation, pegylation, glycosylation, phosphorylation, or substitution (e.g., of the amino group, the carboxylic acid group, one or more protons, or the hydroxyl group) as compared to the general structure. In some embodiments, such modification may, for example, alter the stability or the circulating half-life of a polypeptide containing the modified amino acid as compared to one containing an otherwise identical unmodified amino acid. In some embodiments, such modification does not significantly alter a relevant activity of a polypeptide containing the modified amino acid, as compared to one containing an otherwise identical unmodified amino acid. In some embodiments, the term “amino acid” may be used to refer to a free amino acid; in some embodiments it may be used to refer to an amino acid residue of a polypeptide, e.g., an amino acid residue within a polypeptide.

Analog: As used herein, the term “analog” generally refers to a substance that shares one or more particular structural features, elements, components, or moieties with a reference substance. In some embodiments, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of operations with) one that generates the reference substance. In some embodiments, an analog is or can be generated through performance of a synthetic process different from that used to generate the reference substance.

Antagonist: As used herein, the term “antagonist” generally refers to an agent, or condition whose presence, level, degree, type, or form is associated with a decreased level or activity of a target. An antagonist may include an agent of any chemical class including, for example, small molecules, polypeptides, nucleic acids, carbohydrates, lipids, metals, or any other entity that shows the relevant inhibitory activity. In some embodiments, an antagonist may be a “direct antagonist” in that it binds directly to its target; in some embodiments, an antagonist may be an “indirect antagonist” in that it exerts its influence by mechanisms other than binding directly to its target; e.g., by interacting with a regulator of the target, so that the level or activity of the target is altered). In some embodiments, an “antagonist” may be referred to as an “inhibitor”.

Antibody: As used herein, the term “antibody” generally refers to a polypeptide that includes canonical immunoglobulin sequence elements sufficient to confer specific binding to a particular target antigen. Intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, or “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, such as on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops (“complement determining regions”) (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. Affinity or other binding attributes of Fc regions for Fc receptors can be modulated through glycosylation or other modification. In some embodiments, antibodies produced or utilized in accordance with the present disclosure include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc. Moreover, the term “antibody” as used herein, can refer in appropriate embodiments (unless otherwise stated or clear from context) to any developed constructs or formats for utilizing antibody structural and functional features in alternative presentation. For example, embodiments, an antibody utilized in accordance with the present disclosure is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it may have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]).

Associated: As used herein, two events or entities are generally “associated” with one another, as that term is used herein, if the presence, level, degree, type or form of one is correlated with that of the other. For example, a particular entity (e.g., polypeptide, genetic signature, metabolite, microbe, etc.) is considered to be associated with a particular disease, disorder, or condition, if its presence, level or form correlates with incidence of or susceptibility to the disease, disorder, or condition (e.g., across a relevant population). In some embodiments, two or more entities are physically “associated” with one another if they interact, directly or indirectly, so that they are or remain in physical proximity with one another. In some embodiments, two or more entities that are physically associated with one another are covalently linked to one another; in some embodiments, two or more entities that are physically associated with one another are not covalently linked to one another but are non-covalently associated, for example by hydrogen bonds, van der Waals interaction, hydrophobic interactions, magnetism, and combinations thereof.

Biological Sample: As used herein, the term “biological sample” generally refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, or excretions; or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate method. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, the term “sample” refers to a preparation that is obtained by processing (e.g., by removing one or more components of or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation or purification of certain components, etc.

Biological Network: As used herein, the term “biological network” generally refers to any network that applies to biological systems, having sub-units (e.g., “nodes”) that are linked into a whole, such as species units linked into a whole web. In some embodiments, a biological network is a protein-protein interaction network (PPI), representing interactions among proteins present in a cell, where proteins are nodes and their interactions are edges. In some embodiments, connections between nodes in a PPI are experimentally verified. In some embodiments, connections between nodes are a combination of experimentally verified a mathematically calculated. In some embodiments, a biological network is a human interactome (a network of experimentally derived interactions that occur in human cells, which includes protein-protein interaction information as well as gene expression and co-expression, cellular co-localization of proteins, genetic information, metabolic and signaling pathways, etc.). In some embodiments, a biological network is a gene regulatory network, a gene co-expression network, a metabolic network, or a signaling network.

Combination Therapy: As used herein, the term “combination therapy” generally refers to a clinical intervention in which a subject is simultaneously exposed to two or more therapeutic regimens (e.g. two or more therapeutic agents). In some embodiments, the two or more therapeutic regimens may be administered simultaneously. In some embodiments, the two or more therapeutic regimens may be administered sequentially (e.g., a first regimen administered prior to administration of any doses of a second regimen). In some embodiments, the two or more therapeutic regimens are administered in overlapping dosing regimens. In some embodiments, administration of combination therapy may involve administration of one or more therapeutic agents or modalities to a subject receiving the other agent(s) or modality. In some embodiments, combination therapy does not necessarily require that individual agents be administered together in a single composition (or even necessarily at the same time). In some embodiments, two or more therapeutic agents or modalities of a combination therapy are administered to a subject separately, e.g., in separate compositions, via separate administration routes (e.g., one agent orally and another agent intravenously), or at different time points. In some embodiments, two or more therapeutic agents may be administered together in a combination composition, or even in a combination compound (e.g., as part of a single chemical complex or covalent entity), via the same administration route, or at the same time.

Comparable: As used herein, the term “comparable” generally refers to two or more agents, entities, situations, sets of conditions, etc., that may not be identical to one another but that are sufficiently similar to permit comparison there between so that conclusions may reasonably be drawn based on differences or similarities observed. In some embodiments, comparable sets of conditions, circumstances, individuals, or populations are characterized by a plurality of substantially identical features and one or a small number of varied features. In various approaches, different degrees of identity may be required in any given circumstance for two or more such agents, entities, situations, sets of conditions, etc. to be considered comparable. For example, in various approaches, sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the phrase “corresponding to” generally refers to a relationship between two entities, events, or phenomena that share sufficient features to be reasonably comparable such that “corresponding” attributes are apparent. For example, in some embodiments, the term may be used in reference to a compound or composition, to designate the position or identity of a structural element in the compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, for purposes of simplicity, residues in a polypeptide are often designated using a canonical numbering system based on a reference related polypeptide, so that an amino acid “corresponding to” a residue at position 190, for example, may not actually be the 190^{th} amino acid in a particular amino acid chain but rather corresponds to the residue found at 190 in the reference polypeptide; various approaches may be used to identify “corresponding” amino acids. For example, various approaches may use different sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides or nucleic acids in accordance with the present disclosure.

Dosing regimen or therapeutic regimen: The terms “dosing regimen” and “therapeutic regimen” may be used to generally refer to a set of unit doses (e.g., more than one) that are administered individually to a subject, which may be separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a beneficial outcome when administered across a relevant population (e.g., is a therapeutic dosing regimen).

Improved, increased or reduced: As used herein, the terms “improved,” “increased,” or “reduced,”, or grammatically comparable comparative terms thereof, generally indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.).

Patient or subject: As used herein, the term “patient” or “subject” generally refers to any organism to which a provided composition is or may be administered, e.g., for experimental, diagnostic, prophylactic, cosmetic, or therapeutic purposes. Some patients or subjects include animals (e.g., mammals such as mice, rats, rabbits, non-human primates, or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose or to treat a disease, disorder, or condition.

Pharmaceutical composition: As used herein, the term “pharmaceutical composition” generally refers to an active agent, formulated together with one or more pharmaceutically acceptable carriers. In some embodiments, the active agent is present in unit dose amounts appropriate for administration in a therapeutic regimen to a relevant subject (e.g., in amounts that have been demonstrated to show a statistically significant probability of achieving a predetermined therapeutic effect when administered), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence or magnitude sufficient to achieve statistical relevance). Various approaches may be used to determine, in a given context, a degree or prevalence of difference that is required or sufficient to achieve such statistical significance.

Pharmaceutically acceptable: As used herein, the phrase “pharmaceutically acceptable” generally refers to those compounds, materials, compositions, or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Prevent or prevention: As used herein, the terms “prevent” or “prevention”, when used in connection with the occurrence of a disease, disorder, or condition, generally refer to reducing the risk of developing the disease, disorder or condition or to delaying onset of one or more characteristics or symptoms of the disease, disorder or condition. Prevention may be considered complete when onset of a disease, disorder or condition has been delayed for a predefined period of time.

Reference: As used herein, the term “reference” generally describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested or determined substantially simultaneously with the testing or determination of interest. In some embodiments, a reference or control is a historical reference or control, optionally embodied in a tangible medium. A reference or control is determined or characterized under comparable conditions or circumstances to those under assessment. Sufficient similarities are present to justify reliance on or comparison to a particular possible reference or control.

Therapeutic agent: As used herein, the phrase “therapeutic agent” generally refers to any agent that elicits a pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, or reduce incidence of one or more symptoms or features of a disease, disorder, or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically effective amount: As used herein, the term “therapeutically effective amount” generally refers to an amount of a substance (e.g., a therapeutic agent, composition, or formulation) that elicits a biological response when administered as part of a therapeutic regimen. In some embodiments, a therapeutically effective amount of a substance is an amount that is sufficient, when administered to a subject suffering from or susceptible to a disease, disorder, or condition, to treat, diagnose, prevent, or delay the onset of the disease, disorder, or condition. The effective amount of a substance may vary depending on such factors as the biological endpoint, the substance to be delivered, the target cell or tissue, etc. For example, the effective amount of compound in a formulation to treat a disease, disorder, or condition is the amount that alleviates, ameliorates, relieves, inhibits, prevents, delays onset of, reduces severity of or reduces incidence of one or more symptoms or features of the disease, disorder or condition. In some embodiments, a therapeutically effective amount is administered in a single dose; in some embodiments, multiple unit doses are required to deliver a therapeutically effective amount.

Treat: As used herein, the terms “treat,” “treatment,” or “treating” generally refer to any method used to partially or completely alleviate, ameliorate, relieve, inhibit, prevent, delay onset of, reduce severity of, or reduce incidence of one or more symptoms or features of a disease, disorder, or condition. Treatment may be administered to a subject who does not exhibit signs of a disease, disorder, or condition. In some embodiments, treatment may be administered to a subject who exhibits early signs of the disease, disorder, or condition, for example, for the purpose of decreasing the risk of developing pathology associated with the disease, disorder, or condition.

Variant: As used herein, the term “variant” generally refers to an entity that shows significant structural identity with a reference entity but differs structurally from the reference entity in the presence or level of one or more chemical moieties as compared with the reference entity. In many embodiments, a variant also differs functionally from its reference entity. Whether a particular entity is properly considered to be a “variant” of a reference entity may be based on its degree of structural identity with the reference entity. Any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) or one or more characteristic pendent moieties so that a variant of the small molecule is one that shares the core structural element and the characteristic pendent moieties but differs in other pendent moieties or in types of bonds present (single vs double, E vs Z, etc.) within the core, a polypeptide may have a characteristic sequence element comprised of a plurality of amino acids having designated positions relative to one another in linear or three-dimensional space or contributing to a particular biological function, a nucleic acid may have a characteristic sequence element comprised of a plurality of nucleotide residues having designated positions relative to on another in linear or three-dimensional space. For example, a variant polypeptide may differ from a reference polypeptide as a result of one or more differences in amino acid sequence or one or more differences in chemical moieties (e.g., carbohydrates, lipids, etc.) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. In some embodiments, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (e.g., residues that participate in a particular biological activity). Furthermore, a variant may have not more than 5, 4, 3, 2, or 1 additions or deletions, and often has no additions or deletions, as compared with the parent. Moreover, any additions or deletions may be fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature.

### Disease Gene Expression Signature (A Response Module)

The present disclosure provides, among other things, a disease gene expression signature that, when reversed (all or in substantial part, e.g., after administration of one or more doses of a therapy), indicates that a subject is responding to a therapy. Such an approach is favorable as compared to other methods, as the presently described methods allow for quantification of response on a molecular level, instead of relying on observing changes in clinical characteristics. Indeed, the present disclosure provides methods and systems that encompass an insight that particular molecular signatures, e.g., expression of particular genes, when modulated to resemble healthy subjects, indicate that a diseased subject is responding to a therapy. In some embodiments, a disease expression signature is a pattern of genes that are differentially expressed in diseased subjects as compared to healthy subjects. The presently described disease expression signatures account for subtle differences between diseased and healthy subjects on a molecular level.

In some embodiments, the present disclosure provides methods and systems that encompass an insight that gene expression indicative of response to therapy is not necessarily derived as between subgroups of subjects suffering from the same disease. That is, for example, within a cohort of subjects suffering from a disease, the present disclosure recognizes that analyzing gene expression differences between one or more subgroups of the cohort of subjects may not lead to a gene expression pattern that indicates whether a subject may or may not respond to therapy or otherwise begin to recover from said disease, disorder, or condition. Instead, in some embodiments, the present disclosure analyzes gene expression as between subgroups of diseased subjects having similar gene expression patterns vs. healthy subjects. By analyzing the differences between diseased subjects and healthy subjects, and by identifying key gene expression targets in the diseased subjects that are different from the healthy subjects and also play an important role in driving response, it is understood (without being bound by theory) that modulating the key differentially expressed genes, a diseased subject's gene expression pattern may resemble that of a healthy subject, and thereby lead to regression of the disease.

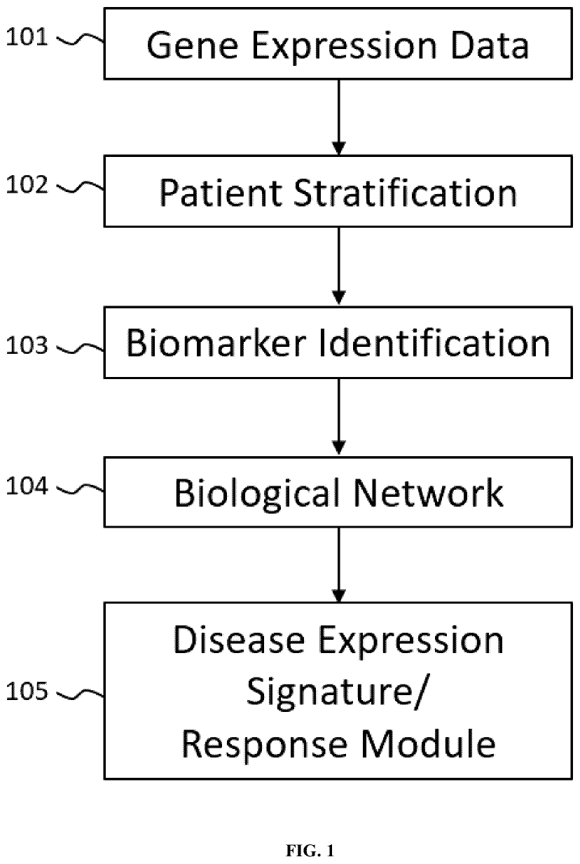

An example workflow for identifying a disease gene expression signature is seen in FIG. 1. In some embodiments, a cohort of gene expression data for a set of subjects suffering from a disease is analyzed (101). Each subject within the cohort is then stratified according to a particular metric (102). For example, in some embodiments, subjects within the cohort are stratified according to whether they are responders or non-responders to a particular therapy (e.g, an anti-TNF therapy). In some embodiments, subjects within the cohort are stratified using supervised or unsupervised clustering algorithms. In some embodiments, subjects within the cohort are stratified using supervised clustering algorithms. In some embodiments, subjects within the cohort are stratified using unsupervised clustering algorithms. In some embodiments, stratifying a cohort of subjects into two or more groups of prior subjects is based on whether the prior subjects do or do not respond to a particular therapy. As used herein, a “therapy” refers to a therapeutic agent as defined here, gene knockout (e.g., making one or more particular genes of a subject inoperative), or gene overexpression (e.g., increasing expression beyond a normal amount of one or more particular genes in a subject).

In some embodiments, baseline expression profiles of the subgroups within the cluster are analyzed and compared to one or more healthy control subjects (103). Genes that are differentially expressed are identified, referred to as “disease candidate genes.” In some embodiments, certain genes that are differentially expressed are selected as “disease candidate genes.” In some embodiments, genes that are significantly differentially expressed are selected to be disease candidate genes. In some embodiments, a significant difference in gene expression is measured by a p-value≤0.05 and absolute fold change of 0.5 or more.

In some embodiments, a disease expression signature comprises all, substantially all or a subset of identified disease candidate genes. In some embodiments, disease candidate genes are optionally mapped onto a biological network (104). Without being bound by theory, it is understood that understanding the connectivity of genes within the disease candidate genes allows for identification of the genes of highest relevance, culling out genes that may not have much of an impact of response when treating a subject for a particular disease. For example, in some embodiments, a biological network is a human interactome map. In some embodiments, genes from the set of disease candidate genes that are either significantly connected or otherwise cluster on a human interactome map are selected to be the disease gene expression signature. In some embodiments, all, substantially all, or a subset of disease candidate genes cluster or are significantly connected on a human interactome map. In some embodiments, a disease gene expression signature comprises disease candidate genes that cluster on a biological network (e.g., a human interactome map). In some embodiments, a disease gene expression signature comprises disease candidate genes that are significantly connected to one another on a biological network (e.g., a human interactome map). In some embodiments, the disease candidate genes are mapped onto a biological network before incorporation into the disease gene expression signature.

In some embodiments, a disease gene expression signature is determined by: analyzing gene expression data from a cohort of subjects suffering from the same disease, disorder, or condition as the subject; stratifying the cohort of subjects into two or more groups of prior subjects based on the gene expression data; and selecting one or more genes having significant differences in gene expression between the two or more groups of prior subjects and a group of healthy subjects (e.g., “disease candidate gene”), to thereby provide the disease gene expression signature.

As used herein, a “healthy gene expression signature” refers to gene expression of response genes in healthy control subjects (e.g., subjects who do not suffer from a disease, disorder, or condition as a subject to be treated as described herein).

In some embodiments, the present disclosure provides a method of determining a disease gene expression signature for quantifying responsiveness to a therapy for subjects suffering from a disease, disorder, or condition, the method comprising: receiving gene expression data from a cohort of subjects suffering from the same disease, disorder, or condition (e.g., suffering from the same disease, disorder, or condition as the subjects being assessed for therapy responsiveness); stratifying the cohort of subjects into two or more groups based on the gene expression data (e.g., grouping subjects from the cohort having similar gene expression); calculating differences in gene expression between the two or more groups of subjects and a group of healthy subjects; and selecting one or more genes having significant differences in gene expression between the two or more groups of subjects and the group of healthy subjects (“disease candidate genes”), to thereby provide the disease gene expression signature.

In some embodiments, the disease candidate genes are mapped onto a biological network (e.g., a human interactome) prior to incorporation into the disease gene expression signature.

In some embodiments, a subset of genes having proximal or spatial relationships on the biological network are selected from the disease candidate genes for incorporation into the disease gene expression signature. In some embodiments, a subset of genes having proximal or spatial relationships on a biological network can be genes having close proximity, for example, on a human interactome map. For example, in some embodiments, genes represented by nodes on a biological network that are connected to two or more nodes are selected, thereby excluding outlier nodes.

In some embodiments, a subset of genes having a significant connection among the disease candidate genes are selected for incorporation into the disease gene expression signature. For example, in some embodiments, a score is assigned for each connection between each node within disease candidate genes. The disease candidate genes can be ranked based on the score, and only the highest ranking disease candidate genes are selected (e.g., the top 10, 20, 30, 40, 50, 60, 70, 80, or 90% of genes from the disease candidate genes).

Accordingly, in some embodiments, the present disclosure provides A method of determining a disease gene expression signature for quantifying responsiveness to a therapy for subjects suffering from a disease, disorder, or condition, the method comprising: receiving gene expression data from a cohort of subjects suffering from the same disease, disorder, or condition (e.g., suffering from the same disease, disorder, or condition as the subjects being assessed for therapy responsiveness); stratifying the cohort of subjects into two or more groups based on the gene expression data (e.g., grouping subjects from the cohort having similar gene expression); calculating differences in gene expression between the two or more groups of subjects and a group of healthy subjects; selecting one or more genes having significant differences in gene expression between the two or more groups of subjects and the group of healthy subjects (“disease candidate genes”); mapping the disease candidate genes onto a biological network (e.g, a human interactome); selecting adjacent genes (e.g., genes on adjacent nodes, for example, on a human interactome map) having significant connection to the disease candidate genes; compiling a list of disease genes comprising the disease candidate genes and adjacent genes; selecting some or all of the genes from the list of disease genes to thereby provide the disease gene expression signature.

In some embodiments, some or all of the genes from the list of disease genes are selected for incorporation into the disease gene expression signature by ranking according to strength of connection to other nodes on the biological network. In some embodiments, the top 10, 20, 30, 40 50, 60 70, 80, or 90% of genes from the list of disease genes are selected for incorporation into the disease gene expression signature.

As described herein, genes of a subject are measured by at least one of a microarray, RNA sequencing, real-time quantitative reverse transcription PCR (qRT-PCR), bead array, ELISA, and protein expression. In some embodiments, gene expression of a subject is measured by subtracting background data, correcting for batch effects, and dividing by mean expression of housekeeping genes. (See Eisenberg & Levanon, “Human housekeeping genes, revisited,” Trends in Genetics, 29(10):569-574 (October 2013), which is incorporated herein by reference for all purposes). In the context of microarray data analysis, background subtraction refers to subtracting the average fluorescent signal arising from probe features on a chip not complimentary to any mRNA sequence, e.g., signals that arise from non-specific binding, from the fluorescence signal intensity of each probe feature. The background subtraction can be performed with different software packages, such as Affymetrix™ Gene Expression Console. Housekeeping genes are involved in basic cell maintenance and, therefore, are expected to maintain constant expression levels in all cells and conditions. The expression level of genes of interest, e.g., those in the response signature, can be normalized by dividing the expression level by the average expression level across a group of selected housekeeping genes. This housekeeping gene normalization procedure calibrates the gene expression level for experimental variability. Further, normalization methods such as robust multi-array average (“RMA”) correct for variability across different batches of microarrays, are available in R packages recommended by either Illumina™ or Affymetrix™ microarray platforms. The normalized data is log transformed, and probes with low detection rates across samples are removed. Furthermore, probes with no available genes symbol or Entrez ID are removed from the analysis.

### Methods of Treatment and Monitoring Therapy

Among other things, the present disclosure provides methods of treating and monitoring therapy of a subject suffering from a disease, disorder, or condition comprising evaluating changes in gene expression within a disease gene expression signature. For example, the present disclosure provides methods and systems that encompass an insight that changes at the molecular level in expression of particular genes within a disease gene expression signature to resemble (all or in part) gene expression of a healthy subject indicate that the subject is responding to therapy, or that the disease is regressing. For example, in some embodiments, the present disclosure provides a method of treating a subject that exhibits a disease gene expression signature, the method comprising administering a therapy determined to revert (or reverse, or otherwise alter) the disease gene expression signature to resemble a healthy gene expression signature.

In some embodiments, the present disclosure provides technologies for validating response to a therapy for a subject from a disease, disorder or condition, comprising analyzing changes in a disease gene expression signature in the subject after administration of the therapy, wherein the disease gene expression signature is determined to quantify responsiveness to the therapy.

Further, the present disclosure provides technologies for monitoring therapy for a given subject or cohort of subjects. As a subject's gene expression level can change over time, it may, in some instances, be necessary or desirable to evaluate a subject at one or more points in time, for example, at specified and or periodic intervals.

In some embodiments, repeated monitoring under time permits or achieves detection of one or more changes in a subject's gene expression profile or characteristics that may impact ongoing treatment regimens. In some embodiments, a change is detected in response to which particular therapy administered to the subject is continued, is altered, or is suspended. In some embodiments, therapy may be altered, for example, by increasing or decreasing frequency or amount of administration of one or more agents or treatments with which the subject is already being treated. Alternatively or additionally, in some embodiments, therapy may be altered by addition of therapy with one or more new agents or treatments. In some embodiments, therapy may be altered by suspension or cessation of one or more particular agents or treatments.

In some embodiments, monitoring comprises quantifying or analyzing changes in a disease gene expression signature. In some embodiments, a disease gene expression signature is determined by analyzing gene expression data from a cohort of subjects suffering from the same disease, disorder, or condition as the subject, stratifying the cohort of subjects into two or more groups of prior subjects based on the gene expression data; and selecting one or more genes having significant differences in gene expression between the two or more groups of prior subjects and a group of healthy subjects (“disease candidate genes”), to thereby provide the disease gene expression signature.

In some embodiments, the present disclosure provides a method of monitoring therapeutic efficacy in a subject suffering from a disease, disorder, or condition, the method comprising monitoring changes in a disease gene expression signature after administration of a therapy, wherein the disease gene expression signature has been derived by a process comprising: analyzing gene expression data from a cohort of subjects suffering from the same disease, disorder, or condition as the subject, stratifying the cohort of subjects into two or more groups based on the gene expression data (e.g., grouping subjects from the cohort having similar gene expression into a group); determining differences in gene expression between the two or more groups of subjects and a group of healthy subjects; and selecting one or more genes having significant differences in gene expression between the two or more groups of subjects and the group of healthy subjects (“disease candidate genes”), to thereby provide the disease gene expression signature.

In some embodiments, stratifying a cohort of prior subjects into two or more groups comprises stratifying subjects based on whether the prior subjects are responders or non-responders to a particular therapy (e.g., an anti-TNF therapy, or a therapy selected from Table 1). In some embodiments, prior subjects are stratified randomly. In some embodiments, prior subjects are stratified by similarities based on gene expression. In some embodiments, similarities based on gene expression in prior subjects are analyzed by a machine learning process.

In some embodiments, a therapy is selected from Table 1.

TABLE 1

| | | | |
|---|---|---|---|
| Seliciclib | AS601245 | Barasertib | MPS-1-IN-1 |
| ALW-II-38-3 | Sigma A6730 | Vemurafenib | XMD-12 |
| ALW-II-49-7 | Sigma A6730 | Enzastaurin | MG-132 |
| AT-7519 | SB 239063 | Enzastaurin | MG-132 |
| AT-7519 | AC220 | NPK76-II-72-1 | Geldanamycin |
| AT-7519 | AC220 | Palbociclib | YM 201636 |
| Tivozanib | WH-4-023 | Palbociclib | FR180204 |
| AZD7762 | WH-4-025 | PF562271 | TWS119 |
| AZD8055 | R406 | PHA-793887 | PF477736 |
| Sorafenib | R406 | KU55933 | Kin237 |
| Sorafenib | BI-2536 | QL-X-138 | Pazopanib |
| Sorafenib | BI-2536 | QL-XI-92 | Pazopanib |
| CP466722 | Motesanib | QL-XII-47 | Pazopanib |
| CP724714 | Motesanib | THZ-2-98-01 | LDN-193189 |
| Alvocidib | KIN001-127 | Torin1 | PF431396 |
| Alvocidib | KIN001-242 | Torin2 | Celastrol |
| GSK429286A | A443654 | KIN001-244 | Amuvatinib |
| GSK461364 | SB590885 | WZ-4-145 | SU11274 |
| GSK461364 | Pictilisib | WZ-7043 | Canertinib |
| GW843682X | Pictilisib | WZ3105 | Canertinib |
| HG-5-113-01 | PD184352 | WZ4002 | SB525334 |
| HG-5-88-01 | PD184352 | XMD11-50 | NVP-AEW541 |
| HG-6-64-01 | PLX-4720 | XMD11-85h | SGX523 |
| Neratinib | PLX-4720 | XMD13-2 | SGX523 |
| Neratinib | AZ-628 | XMD14-99 | MGCD265 |
| JW-7-24-1 | Lapatinib | XMD15-27 | analog |
| Dasatinib | Lapatinib | XMD16-144 | PHA-665752 |
| Dasatinib | Sirolimus | JWE-035 | PHA-665752 |
| Tozasertib | ZSTK474 | XMD8-85 | PI103 |
| Tozasertib | AS605240 | XMD8-92 | PI103 |
| GNF2 | BX-912 | ZG-10 | PI103 |
| Imatinib | Selumetinib | ZM-447439 | Dovitinib |
| Imatinib | Selumetinib | Erlotinib | Dovitinib |
| NVP-TAE684 | MK2206 | Erlotinib | GSK 690693 |
| NVP-TAE684 | CG-930 | Erlotinib | GSK 690693 |
| CGP60474 | AZD-6482 | Gefitinib | Ibrutinib |
| PD173074 | TAK-715 | Gefitinib | Masitinib |
| Crizotinib | NU7441 | Nilotinib | Masitinib |
| Crizotinib | GSK1070916 | Nilotinib | Tivantinib |
| BMS345541 | OSI-027 | JNK-9L | SNS-032 |
| BMS345541 | OSI-027 | PD0325901 | SNS-032 |
| GW-5074 | WYE-125132 | Taxol | Afatinib |
| KIN001-042 | KIN001-220 | Taxol | Afatinib |
| KIN001-043 | MLN8054 | Staurosporine | GSK1904529A |
| Saracatinib | MLN8054 | Staurosporine | Linsitinib |
| KIN001-055 | Barasertib | RO-3306 | TPCA-1 |
| Ruxolitinib | PI3K-IN-1 | NVP-TAE226 | BMS509744 |
| Ruxolitinib | A 769662 | JNK-IN-5A | Docetaxel |
| Ruxolitinib | Sunitinib | BMS-536924 | Doxorubicin |
| AZD-1480 | Sunitinib | Go 6976 | Doxorubicin |
| Momelotinib | Sunitinib | Go-6983 | Epirubicin |
| Momelotinib | Y-27632 | KIN001-021 | Etoposide |
| Fedratinib | Brivanib | KIN001-111 | Etoposide |
| Fedratinib | Brivanib | KIN001-123 | Fascaplysin |
| Trametinib | OSI-930 | KIN001-135 | Gemcitabine |
| Trametinib | ABT-737 | KN-93 | Gemcitabine |
| BMS 777607 | ABT-737 | S-Trityl-L- | Glycyl-H-1152 |
| Olaparib | CHIR-99021 | cysteine | GSK1838705A |
| Veliparib | GDC-0879 | SU6656 | GSK1838705A |
| Omipalisib | GDC-0879 | U-0126 | GSK923295 |
| Buparlisib | Linifanib | PKC412 | Ibandronate |
| XL147 | Linifanib | PKC412 | ICRF-193 |
| Y39983 | BGJ398 | GSK2334470 | Ispinesib |
| Ponatinib | Rigosertib | Dacomitinib | Ixabepilone |
| Nintedanib | Rigosertib | AG1478 | L-779450 |
| Nintedanib | CC-401 | AST1306 | LBH589 |
| MK 1775 | Chelerythrine | Regorafenib | Methotrexate |
| KIN001-266 | Ki20227 | Tofacitinib | Methotrexate |
| AT7867 | Ki20227 | Tofacitinib | Pevonedistat |
| KU-60019 | BX795 | Tofacitinib | Pevonedistat |
| JNJ38877605 | Bosutinib | EO1428 | NSC 663284 |
| Foretinib | Bosutinib | IKK16 | NU6102 |
| Foretinib | PIK-93 | KU63794 | Nutlin 3a |
| AZD 5438 | HMN-214 | Lestaurtinib | Oxaliplatin |
| Pelitinib | KW2449 | Lestaurtinib | Oxamflatin |
| SB 216763 | KW2449 | PF-3758309 | PD 98059 |
| Luminespib | Kin236 | Dactolisib | Pemetrexed |
| SP600125 | Cabozantinib | Alpelisib | Purvalanol A |
| BIX 02189 | KIN001-269 | GDC-0980 | SB-3 CT |
| AZD8330 | KIN001-270 | Everolimus | (Z)-4- |
| PF04217903 | KIN001-260 | 17-AAG | Hydroxy- |
| BAY61-3606 | Vandetanib | 17-AAG | tamoxifen |
| BAY61-3606 | Vandetanib | 5-DFUR | TCS 2312 |
| SB 203580 | PF 573228 | 5-FU | Temsirolimus |
| SB 203580 | NVP-BHG712 | AG1024 | Topotecan |
| VX-745 | CH5424802 | AS-252424 | Topotecan |
| VX-745 | D 4476 | Bortezomib | Trichostatin A |
| Doramapimod | A66 | Carboplatin | Triciribine |
| Doramapimod | CAL-101 | CGC-11047 | Triciribine |
| JNJ 26854165 | INK-128 | CGC-11144 | Vinorelbine |
| TGX221 | RAF 265 | Cisplatin | Vinorelbine |
| GSK1059615 | RAF 265 | Cisplatin | Vorinostat |
| PHA-767491 | I-BET | CPT-11 | XRP44X |
| BS-181 | I-BET151 | Ganetespib | Dabrafenib |
| Dinaciclib | Ischemin | GDC-0994 | PYR41 |
| SGI-1776 | UNC669 | GSK2636771 | CID755673 |
| AZD4547 | UNC1215 | KX01 | VX-11e |
| BMS-754807 | IOX2 | LY2090314 | BI-D1870 |
| Shikonin | Epigallocatechin | LY-2584702 | ML-7 |
| Mitomycin C | gallate | NMS-1286937 | PIM12 kinase |
| Thapsigargin | OTSSP167 | Pacritinib | inhibitor V |
| Thapsigargin | Ipatasertib | P529 | Barasertib |
| Embelin | CX-5461 | PF-06463922 | BMX-IN-1 |
| IPA-3 | HG-9-91-01 | SR-2516 | Spebrutinib |
| Bryostatin 1 | HG-14-8-02 | S-Ruxolitinib | THZ1 |
| NSC-87877 | HG-14-10-04 | Tideglusib | THZ1 |
| LFM-A13/DDE- | Baicalein | Volasertib | GNE7915 |
| 28 | Olomoucine II | XL019 | BIX02188 |
| GSK650394 | Torkinib | XL413 | WZ4003 |
| Azacitidine | Torkinib | Abemaciclib | BIX 02565 |
| Decitabine | Torkinib | Alisertib | LY2109761 |
| RG-108 | Valproic acid | ALK-IN-1 | AZD2014 |
| Iniparib | Z-Leu-Leu- | AT9283 | Ralimetinib |
| Rucaparib | Norvalinal | Ceritinib | PH-797804 |
| JW55 | NVP-BGT226 | Ribociclib | VX-702 |
| C646 | (s)-CR8 | LY2874455 | SB202190 |
| Garcinol | DCC-2036 | Poziotinib | SCH772984 |
| Anacardic acid | ABT-751 | CGP 57380 | Axitinib |
| CTB | Enzalutamide | Dorsomorphin | Cediranib |
| Belinostat | Baricitinib | FRAX597 | Taselisib |
| Entinostat | CGP74514A | GW2580 | CH5183284 |
| Mocetinostat | 5z-7-oxozeaenol | Losmapimod | EW-7197 |
| Pracinostat | XL765 | Necrostatin-1 | Riviciclib |
| MC1568 | AZ 20 | PF-4708671 | NH125 |
| Rocilinostat | CGK733 | PP1 | SAL003 |
| Selisistat | NU7026 | PRT062607 | (−)-Blebbistatin |
| AGK2 | VE-821 | RO 31-8220 | SKI II |
| Resveratrol | LY2603618 | Sotrastaurin | URMC-099 |
| BIX-01294 | JNK-IN-8 | TAK-632 | Staurosporine |
| UNC0638 | MRT67307 | Ellagic acid | aglycone |
| GSK-J1 | GNF-5837 | H89 | IP6K/IP3K |
| GSK-J2 | CP-673451 | KN62 | inhibitor |
| GSK-J4 | Navitoclax | KRN633 | ABT-702 |
| Daminozide | ASP3026 | Leflunomide | AG-F-89549 |
| Methylstat | AZD1208 | TG003 | AX20017 |
| Tranylcypromine | AZD5363 | Febuxostat | BAY-11-7082 |
| PFI-1 | CUDC-907 | GW 1516 | Bohemine |
| (+)-JQ1 | Entospletinib | Lenalidomide | CGP-029482 |
| (−)-JQ1 | Filgotinib | NG25 | GTPL5944 |
| H-8 | acid | 5-(4- | GTPL6019 |
| JNJ-10198409 | Celecoxib | fluorophenyl)-3- | GTPL6027 |
| RGB-286147 | Chk2 inhibitor II | hydroxy-4-(5- | Senexin B |
| ML-9 | Chloroquine | methyl-2-furoyl)- | BMS-265246 |
| R59949 | Dichloroacetate | 1-(3- | HY-17541A |
| SCH 51344 | Disulfiram | pyridinylmethyl)- | SJB2-043 |
| ST50842732 | FTase Inhibitor I | 1,5-dihydro-2H- | 1247825-37-1 |
| TBCA | GM6001 | pyrrol-2-one | HY-50737A |
| TX-1918 | LY294002 | Pimozide | HY-50736 |
| R 59-022 | Mebendazole | GW7647 | ML-323 |
| PF 3644022 | Methylglyoxal | MI-2 | USP7-IN-1 |
| JNK-IN-11 | Nelfinavir | Sepantronium | HBX19818 |
| A-1210477 | PS-1145 | HBX 41108 | HY-17542 |
| Mitoxantrone | QNZ | Doxycycline | z-VAE(OMe)- |
| Radicicol | Ribavirin | Degrasyn | fmk |
| Withaferin A | Ro 32-0432 | SJB3-019A | PB49673382 |
| Bleomycin | Sulindac sulfide | IU1 | SB1-F-21 |
| Brefeldin A | TAPI-0 | Spautin-1 | SB1-F-22 |
| Cycloheximide | TCS PIM-1 1 | Vialinin A | THZ531 |
| Fluvastatin | ERK5-IN-1 | Kenpaullone | QL-IV-100 |
| Monensin | b-AP15 | Mevastatin | QL-V-107 |
| Vincristine | STK547622 | Defactinib | QL-V-73 |
| Dactinomycin | LDN57444 | SHP099 | QL-VI-86 |
| 2-deoxyglucose | P22077 | Ulixertinib | QL-VIII-58 |
| Bromopyruvic | Trifluoperazine | LY3023414 | QL-XII-108 |
| | | AZD6738 | QL-XII-61 |

In some embodiments, a therapy is an anti-TNF therapy. In some embodiments, an anti-TNF therapy is selected from infliximab, etanercept, adalimumab, certolizumab pegol, golimumab, and biosimilars thereof. In some embodiments, an anti-TNF therapy is infliximab. In some embodiments, an anti-TNF therapy is etanercept. In some embodiments, an anti-TNF therapy is adalimumab. In some embodiments, an anti-TNF therapy is certolizumab pegol. In some embodiments, an anti-TNF therapy is golimumab. In some embodiments, an anti-TNF therapy is a biosimilar of infliximab, etanercept, adalimumab, certolizumab pegol, or golimumab.

In some embodiments, a therapy is selected from rituximab, sarilumab, tofacitinib citrate, lefunomide, vedolizumab, tocilizumab, anakinra, and abatacept. In some embodiments, a therapy is rituximab. In some embodiments, a therapy is sarilumab. In some embodiments, a therapy is tofacitinib citrate. In some embodiments, a therapy is lefunomide. In some embodiments, a therapy is vedolizumab. In some embodiments, a therapy is tocilizumab. In some embodiments, a therapy is anakinra. In some embodiments, a therapy is abatacept.

In some embodiments, a disease, disorder, or condition comprises ulcerative colitis, Crohn's disease, rheumatoid arthritis, juvenile arthritis, psoriatic arthritis, plaque psoriasis, or ankylosing spondylitis. In some embodiments, a disease, disorder, or condition is ulcerative colitis. In some embodiments, a disease, disorder, or condition is Crohn's disease. In some embodiments, a disease, disorder, or condition is rheumatoid arthritis. In some embodiments, a disease, disorder, or condition comprises ulcerative colitis, Crohn's disease, rheumatoid arthritis, juvenile arthritis, psoriatic arthritis, plaque psoriasis, or ankylosing spondylitis.

### Patient Stratification and Trial Design

The present disclosure further provides methods and systems that encompass an insight that changes in gene expression on the molecular level can occur faster and are easily quantifiable as compared to changes in clinical characteristics in a subject who has received a therapy. For example, the present disclosure provides methods and systems that encompass an insight that responsiveness of patients to therapy can be quantified early in a dosing regimen, allowing practitioners to alter treatment course in individual subjects, or otherwise suspend treatment for subjects, including in large scale studies, e.g., in clinical trials. Such measures allow study designers to identify which subjects are not responding to therapy on the basis of individual biology, and remove them from the study, preventing risking potential harm to any non-responsive subject, as well as saving time and resources for the study designers.

Accordingly, in some embodiments, the present disclosure provides methods and systems that encompass a method of identifying and selecting subjects for a clinical trial comprising receiving gene expression data of a cohort subjects; analyzing the gene expression data to detect the presence of a disease gene expression signature; administering at least one dose of a therapy to the subjects; identifying changes in the disease gene expression signature relative to gene expression of a healthy subject; selecting subjects for the clinical trial who exhibit a quantifiable change in the disease gene expression signature towards gene expression of a healthy subject.

### Systems and Architecture

Also described herein is a method for engineering a personalized therapy for a subject, the method comprising: receiving or generating a disease gene expression signature comprising a set of response genes; receiving or generating of the computing device, a set of one or more potential therapies that alter expression of the one or more response genes; ranking each of the set of the one or more potential therapies according to significance of alteration of the one or more response genes, to provide a set of one or more candidate therapies; determining one or more potential targets directly modulated by the set of one or more candidate therapies, optionally by mapping the one or more potential targets onto a biological network; ranking significance of connectivity between each of the one or more potential targets and the set of response genes; selecting a target for treatment from the one or more potential targets; and selecting the personalized therapy that modulates the target for treatment.

In some embodiments, a disease gene expression signature is determined by: receiving or generating gene expression data from a cohort of subjects suffering from the same disease, disorder, or condition as the subject; stratifying the cohort of subjects into two or more groups of prior subjects based on the gene expression data; and selecting one or more genes having significant differences in gene expression between the two or more groups of prior subjects and a group of healthy subjects (“disease candidate genes”), to thereby provide the disease gene expression signature.

In some embodiments, disease candidate genes are mapped onto a biological network before being selected to be part of the disease gene expression signature.

In some embodiments, determining one or more potential targets further comprises mapping targets of the one or more candidate therapies onto a biological network, and selecting potential targets based on topological information provided by to the biological network.

In some embodiments, ranking of each of the one or more potential therapies comprises: calculating a difference in expression level of the set of response genes after treatment with the one or more potential therapies relative to the set of response genes before treatment with the one or more potential therapies; and calculating a p-value for each of the one or more potential therapies.

In some embodiments, potential targets are identified by a machine-learning process.

In some embodiments, a machine-learning process comprises a random walk.

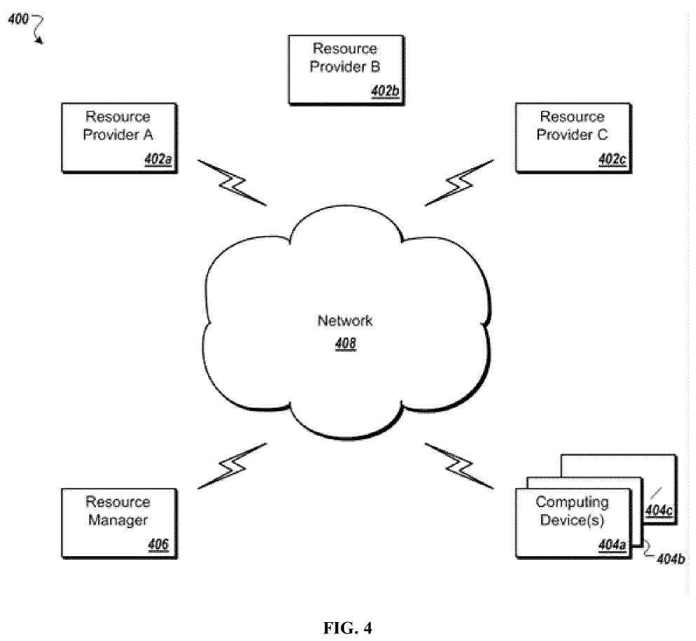

As shown in FIG. 4, an implementation of a network environment 400 for use in providing systems, methods, and architectures as described herein is shown and described. In brief overview, referring now to FIG. 4, a block diagram of an exemplary cloud computing environment 400 is shown and described. The cloud computing environment 400 may include one or more resource providers 402a, 402b, 402c (collectively, 402). Each resource provider 402 may include computing resources. In some implementations, computing resources may include any hardware or software used to process data. For example, computing resources may include hardware or software capable of executing algorithms, computer programs, or computer applications. In some implementations, exemplary computing resources may include application servers or databases with storage and retrieval capabilities. Each resource provider 402 may be connected to any other resource provider 402 in the cloud computing environment 400. In some implementations, the resource providers 402 may be connected over a computer network 408. Each resource provider 402 may be connected to one or more computing device 404a, 404b, 404c (collectively, 404), over the computer network 408.

The cloud computing environment 400 may include a resource manager 406. The resource manager 406 may be connected to the resource providers 402 and the computing devices 404 over the computer network 408. In some implementations, the resource manager 406 may facilitate the provision of computing resources by one or more resource providers 402 to one or more computing devices 404. The resource manager 406 may receive a request for a computing resource from a particular computing device 404. The resource manager 406 may identify one or more resource providers 402 capable of providing the computing resource requested by the computing device 404. The resource manager 406 may select a resource provider 402 to provide the computing resource. The resource manager 406 may facilitate a connection between the resource provider 402 and a particular computing device 404. In some implementations, the resource manager 406 may establish a connection between a particular resource provider 402 and a particular computing device 404. In some implementations, the resource manager 406 may redirect a particular computing device 404 to a particular resource provider 402 with the requested computing resource.

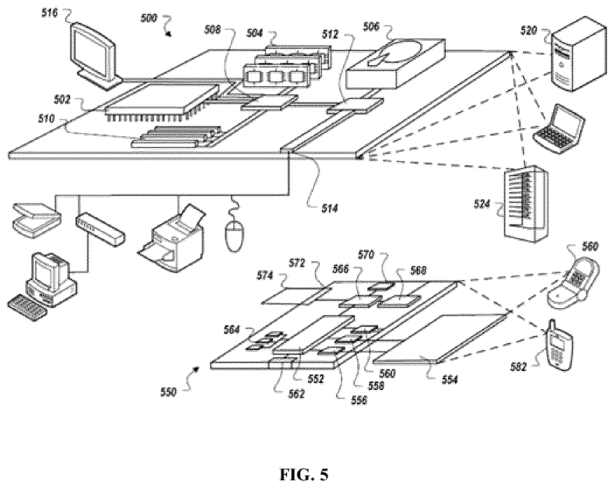

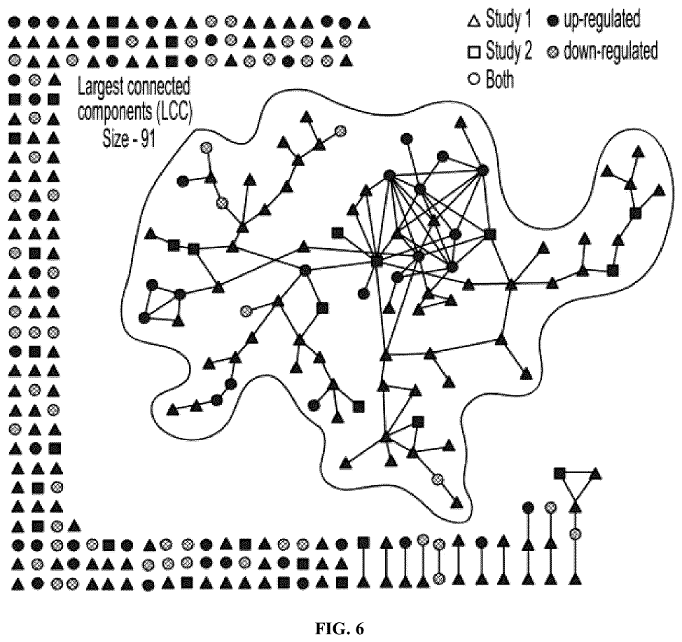

FIG. 5 shows an example of a computing device 500 and a mobile computing device 550 that can be used to implement the techniques described herein. The computing device 500 is intended to represent various forms of digital computers, such as laptops, desktops, workstations, personal digital assistants, servers, blade servers, mainframes, and other appropriate computers. The mobile computing device 550 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 500 includes a processor 502, a memory 504, a storage device 506, a high-speed interface 508 connecting to the memory 504 and multiple high-speed expansion ports 510, and a low-speed interface 512 connecting to a low-speed expansion port 514 and the storage device 506. Each of the processor 502, the memory 504, the storage device 506, the high-speed interface 508, the high-speed expansion ports 510, and the low-speed interface 512, are interconnected using various busses, and may be mounted on a common motherboard or in other manners as appropriate. The processor 502 can process instructions for execution within the computing device 500, including instructions stored in the memory 504 or on the storage device 506 to display graphical information for a GUI on an external input/output device, such as a display 516 coupled to the high-speed interface 508. In other implementations, multiple processors or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 504 stores information within the computing device 500. In some implementations, the memory 504 is a volatile memory unit or units. In some implementations, the memory 504 is a non-volatile memory unit or units. The memory 504 may also be another form of computer-readable medium, such as a magnetic or optical disk.

The storage device 506 is capable of providing mass storage for the computing device 500. In some implementations, the storage device 506 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 502), perform one or more methods, such as those described above. The instructions can also be stored by one or more storage devices such as computer- or machine-readable mediums (for example, the memory 504, the storage device 506, or memory on the processor 502).

The high-speed interface 508 manages bandwidth-intensive operations for the computing device 500, while the low-speed interface 512 manages lower bandwidth-intensive operations. Such allocation of functions is an example only. In some implementations, the high-speed interface 508 is coupled to the memory 504, the display 516 (e.g., through a graphics processor or accelerator), and to the high-speed expansion ports 510, which may accept various expansion cards (not shown). In the implementation, the low-speed interface 512 is coupled to the storage device 506 and the low-speed expansion port 514. The low-speed expansion port 514, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 500 may be implemented in a number of different forms, as shown in FIG. 5. For example, it may be implemented as a standard server 520, or multiple times in a group of such servers. In addition, it may be implemented in a personal computer such as a laptop computer 522. It may also be implemented as part of a rack server system 524. Alternatively, components from the computing device 500 may be combined with other components in a mobile device (not shown), such as a mobile computing device 550. Each of such devices may contain one or more of the computing device 500 and the mobile computing device 550, and an entire system may be made up of multiple computing devices communicating with each other.

The mobile computing device 550 includes a processor 552, a memory 564, an input/output device such as a display 554, a communication interface 566, and a transceiver 568, among other components. The mobile computing device 550 may also be provided with a storage device, such as a micro-drive or other device, to provide additional storage. Each of the processor 552, the memory 564, the display 554, the communication interface 566, and the transceiver 568, are interconnected using various buses, and several of the components may be mounted on a common motherboard or in other manners as appropriate.

The processor 552 can execute instructions within the mobile computing device 550, including instructions stored in the memory 564. The processor 552 may be implemented as a chipset of chips that include separate and multiple analog and digital processors. The processor 552 may provide, for example, for coordination of the other components of the mobile computing device 550, such as control of user interfaces, applications run by the mobile computing device 550, and wireless communication by the mobile computing device 550.

The processor 552 may communicate with a user through a control interface 558 and a display interface 556 coupled to the display 554. The display 554 may be, for example, a TFT (Thin-Film-Transistor Liquid Crystal Display) display or an OLED (Organic Light Emitting Diode) display, or other appropriate display technology. The display interface 556 may comprise appropriate circuitry for driving the display 554 to present graphical and other information to a user. The control interface 558 may receive commands from a user and convert them for submission to the processor 552. In addition, an external interface 562 may provide communication with the processor 552, so as to enable near area communication of the mobile computing device 550 with other devices. The external interface 562 may provide, for example, for wired communication in some implementations, or for wireless communication in other implementations, and multiple interfaces may also be used.

The memory 564 stores information within the mobile computing device 550. The memory 564 can be implemented as one or more of a computer-readable medium or media, a volatile memory unit or units, or a non-volatile memory unit or units. An expansion memory 574 may also be provided and connected to the mobile computing device 550 through an expansion interface 572, which may include, for example, a SIMM (Single In Line Memory Module) card interface. The expansion memory 574 may provide extra storage space for the mobile computing device 550, or may also store applications or other information for the mobile computing device 550. Specifically, the expansion memory 574 may include instructions to carry out or supplement the processes described above, and may include secure information also. Thus, for example, the expansion memory 574 may be provide as a security module for the mobile computing device 550, and may be programmed with instructions that permit secure use of the mobile computing device 550. In addition, secure applications may be provided via the SIMM cards, along with additional information, such as placing identifying information on the SIMM card in a non-hackable manner.

The memory may include, for example, flash memory or NVRAM memory (non-volatile random access memory), as discussed below. In some implementations, instructions are stored in an information carrier, that the instructions, when executed by one or more processing devices (for example, processor 552), perform one or more methods, such as those described above. The instructions can also be stored by one or more storage devices, such as one or more computer- or machine-readable mediums (for example, the memory 564, the expansion memory 574, or memory on the processor 552). In some implementations, the instructions can be received in a propagated signal, for example, over the transceiver 568 or the external interface 562.

The mobile computing device 550 may communicate wirelessly through the communication interface 566, which may include digital signal processing circuitry where necessary. The communication interface 566 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 568 using a radio-frequency. In addition, short-range communication may occur, such as using a Bluetooth®, Wi-Fi™, or other such transceiver (not shown). In addition, a GPS (Global Positioning System) receiver module 570 may provide additional navigation- and location-related wireless data to the mobile computing device 550, which may be used as appropriate by applications running on the mobile computing device 550.

The mobile computing device 550 may also communicate audibly using an audio codec 560, which may receive spoken information from a user and convert it to usable digital information. The audio codec 560 may likewise generate audible sound for a user, such as through a speaker, e.g., in a handset of the mobile computing device 550. Such sound may include sound from voice telephone calls, may include recorded sound (e.g., voice messages, music files, etc.) and may also include sound generated by applications operating on the mobile computing device 550.

The mobile computing device 550 may be implemented in a number of different forms, as shown in FIG. 5. For example, it may be implemented as a cellular telephone 580. It may also be implemented as part of a smart-phone 582, personal digital assistant, or other similar mobile device.

Various implementations of the systems and techniques described here can be realized in digital electronic circuitry, integrated circuitry, specially designed ASICs (application specific integrated circuits), computer hardware, firmware, software, or combinations thereof. These various implementations can include implementation in one or more computer programs that are executable or interpretable on a programmable system including at least one programmable processor, which may be special or general purpose, coupled to receive data and instructions from, and to transmit data and instructions to, a storage system, at least one input device, and at least one output device.

These computer programs (e.g., as programs, software, software applications or code) include machine instructions for a programmable processor, and can be implemented in a high-level procedural or object-oriented programming language, or in assembly/machine language. As used herein, the terms machine-readable medium and computer-readable medium refer to any computer program product, apparatus or device (e.g., magnetic discs, optical disks, memory, Programmable Logic Devices (PLDs)) used to provide machine instructions or data to a programmable processor, including a machine-readable medium that receives machine instructions as a machine-readable signal. The term machine-readable signal refers to any signal used to provide machine instructions or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server may be remote from each other and may interact through a communication network. The relationship of client and server arises by virtue of computer programs running on the respective computers and having a client-server relationship to each other.

In some implementations, the modules described herein can be separated, combined or incorporated into single or combined modules. The modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein. In view of the structure, functions and apparatus of the systems and methods described here, in some implementations.

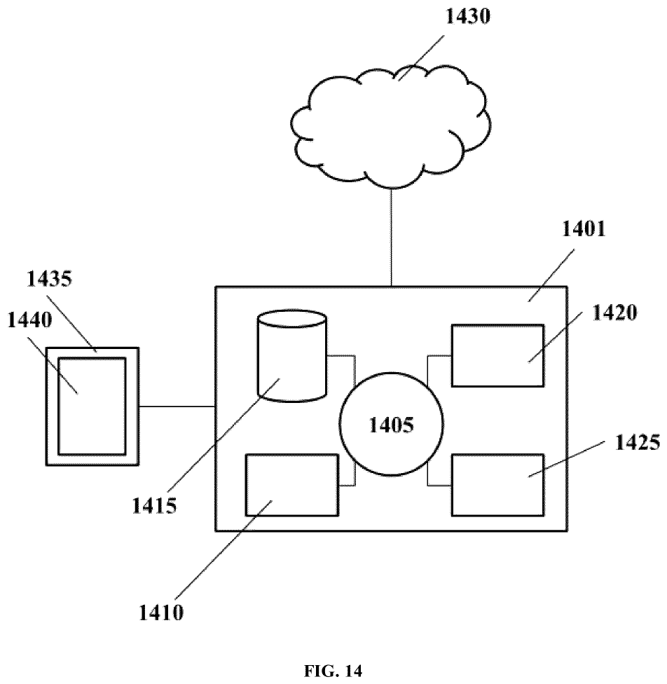

The present disclosure provides computer systems that are programmed to implement methods of the disclosure. FIG. 14 shows a computer system 1401 that is programmed or otherwise configured to perform analysis or operations of various methods. The computer system 1401 can regulate various aspects of methods and systems of the present disclosure, such as, for example, perform an algorithm, analyze data, or output results of an algorithm. The computer system 1401 can be an electronic device of a user or a computer system that is remotely located with respect to the electronic device. The electronic device can be a mobile electronic device.

The computer system 1401 includes a central processing unit (CPU, also “processor” and “computer processor” herein) 1405, which can be a single core or multi core processor, or a plurality of processors for parallel processing. The computer system 1401 also includes memory or memory location 1410 (e.g., random-access memory, read-only memory, flash memory), electronic storage unit 1415 (e.g., hard disk), communication interface 1420 (e.g., network adapter) for communicating with one or more other systems, and peripheral devices 1425, such as cache, other memory, data storage and/or electronic display adapters. The memory 1410, storage unit 1415, interface 1420 and peripheral devices 1425 are in communication with the CPU 1405 through a communication bus (solid lines), such as a motherboard. The storage unit 1415 can be a data storage unit (or data repository) for storing data. The computer system 1401 can be operatively coupled to a computer network (“network”) 1430 with the aid of the communication interface 1420. The network 1430 can be the Internet, an internet and/or extranet, or an intranet and/or extranet that is in communication with the Internet. The network 1430 in some cases is a telecommunication and/or data network. The network 1430 can include one or more computer servers, which can enable distributed computing, such as cloud computing. The network 1430, in some cases with the aid of the computer system 1401, can implement a peer-to-peer network, which may enable devices coupled to the computer system 1401 to behave as a client or a server.

The CPU 1405 can execute a sequence of machine-readable instructions, which can be embodied in a program or software. The instructions may be stored in a memory location, such as the memory 1410. The instructions can be directed to the CPU 1405, which can subsequently program or otherwise configure the CPU 1405 to implement methods of the present disclosure. Examples of operations performed by the CPU 1405 can include fetch, decode, execute, and writeback.

The CPU 1405 can be part of a circuit, such as an integrated circuit. One or more other components of the system 1401 can be included in the circuit. In some cases, the circuit is an application specific integrated circuit (ASIC).

The storage unit 1415 can store files, such as drivers, libraries and saved programs. The storage unit 1415 can store user data, e.g., user preferences and user programs. The computer system 1401 in some cases can include one or more additional data storage units that are external to the computer system 1401, such as located on a remote server that is in communication with the computer system 1401 through an intranet or the Internet.

The computer system 1401 can communicate with one or more remote computer systems through the network 1430. For instance, the computer system 1401 can communicate with a remote computer system of a user (e.g., a medical professional or patient). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1401 via the network 1430.

Methods as described herein can be implemented by way of machine (e.g., computer processor) executable code stored on an electronic storage location of the computer system 1401, such as, for example, on the memory 1410 or electronic storage unit 1415. The machine executable or machine readable code can be provided in the form of software. During use, the code can be executed by the processor 1405. In some cases, the code can be retrieved from the storage unit 1415 and stored on the memory 1410 for ready access by the processor 1405. In some situations, the electronic storage unit 1415 can be precluded, and machine-executable instructions are stored on memory 1410.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1401 can include or be in communication with an electronic display 1435 that comprises a user interface (UI) 1440 for providing, for example, an input or output of data, or an visual output relating to an algorithm. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1405. The algorithm can, for example, perform analysis or operations of methods of the present disclosure.

### EXAMPLES

The following non-limiting examples are intended to illustrate various embodiments of the subject matter described herein.

### Example 1—Systemic Bioinformatic and Network-Based Analysis of Ulcerative Colitis

Gene expression data of eight ulcerative colitis (UC) patient cohorts that went through anti-TNF therapy where downloaded and studied in two separate batches (Study 1 and 2 described in Tables 2 and 3, respectively).

TABLE 2

| | |
|---|---|
| Discovery cohort: | GSE16879, GSE23597, GSE38713, GSE12251, |
| | GSE13367, GSE36807, GSE47908 |
| Assay | Affymetrix ™ Human Genome U133 Plus 2.0 |
| | Array microarray |
| # of Healthy | 41 |
| # of UC active | 169 (R: 40, NR: 39) |

TABLE 3

| | |
|---|---|
| Discovery cohort: | GSE92415 |
| Assay | Affymetrix ™ HT HG-U133 + microarray |
| # of Healthy | 21 |
| # of UC active | 87 (R: 32, NR: 27) |

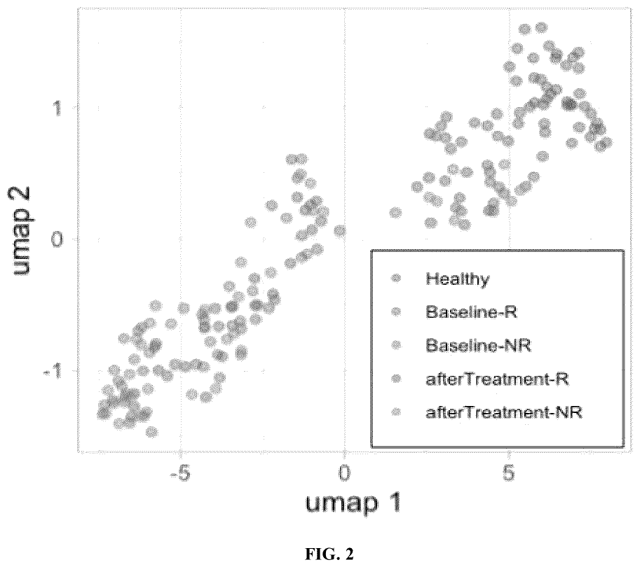

Gene expression profile of responders and non-responders to treatment at baseline and after treatment when compared to each other and to healthy controls (FIG. 2). Analysis shows that molecular signatures of responders to treatment (after treatment) resemble healthy controls.

Molecular differences of a specific disease subpopulations are subtle. Comparing baseline expression profiles of UC responders and non-responders does not reveal any significantly differentiated genes. Instead, molecular differences of patient subpopulations are more pronounced when compared to healthy controls.

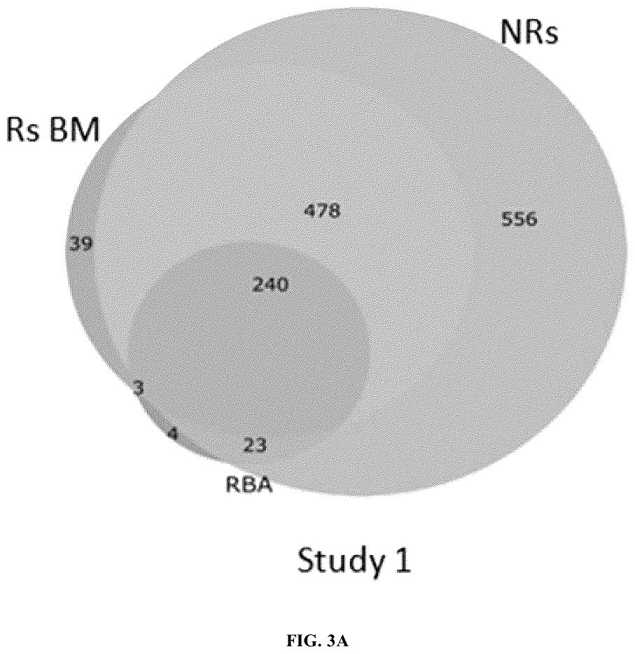

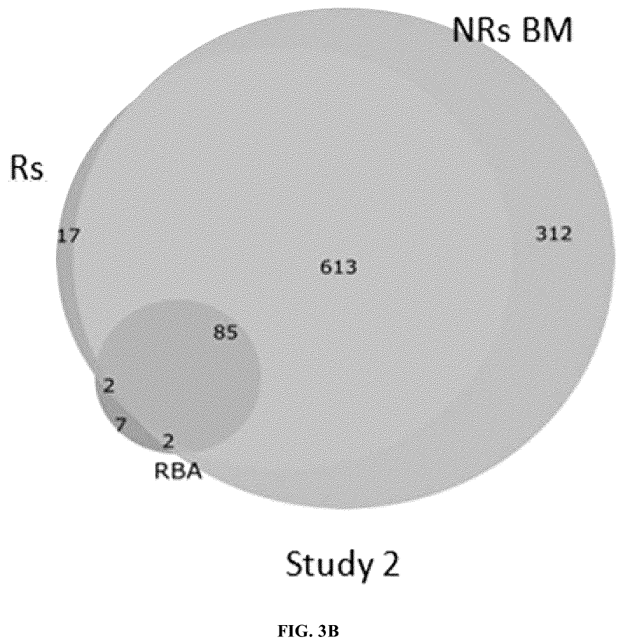

Gene expression of non-responders were derived by comparing the baseline expression profile of non-responders to healthy controls. The inverse was also performed (e.g., comparing baseline expression profile of responders to healthy controls). Both studies showed that responder biomarker set is almost fully contained within non-responders' biomarker set and non-responder biomarker set was generally twice as large as responder biomarker set, potentially suggesting a more severe disease state for non-responders (FIGS. 3A and 3B).

FIG. 1 shows an example workflow for identification of a disease gene expression signature (also referred to herein as a response module).

For example, in some embodiments, in response module discovery, biomarkers associated to specific patient subpopulations are identified as compared to healthy controls. In order to achieve molecular remission e.g., making patient's transcriptomics resemble healthy controls, a desirable downstream effect is identified, where the response module genes are reversed.

Subjects were be stratified using both supervised and unsupervised clustering algorithms. To identify subject subpopulation biomarkers, baseline expression profile of different patient subpopulations was compared to healthy controls. These biomarkers are then mapped on the map of Human Interactome. It was found that identified biomarkers form a significant cluster on the network e.g., the nodes are not scattered and instead are significantly interacting with each other forming a subnetwork consisting subpopulation-specific biomarkers (response module). It was also discovered that after-treatment expression profile of patients who responded to treatment resemble healthy controls and so response to treatment can be translated to reverting the response module genes to make them resemble healthy controls.

### Example 2—A Validated Systems-Based Multi-Omic Data Analytics Platform to Identify Novel Drug Targets in Ulcerative Colitis

Tumor necrosis factor-α inhibitors (TNFi) have been a standard treatment in ulcerative colitis (UC) for nearly 20 years. However, not every patient responds to TNFi therapies, in citing development of alternative UC treatments. Disclosed herein are multi-omic network biology methods for prioritization of protein targets for UC treatment. Disclosed methods may identify network modules on a Human Interactome comprising genes contributing to a predisposition to UC (a Genotype module), genes whose expression may be altered to achieve low disease activity (a Response module), and proteins whose perturbation may alter expression of the Response module genes in a favorable direction (a Treatment module). Targets may be prioritized based on their topological relevance to the Genotype module and functional similarity to the Treatment module. In an example, methods described herein in UC may efficiently recover protein targets associated with launched and underdevelopment drugs for UC treatment. Avenues may be enabled for finding novel and repurposing therapeutic opportunities in UC and other complex diseases.

### Introduction

Ulcerative colitis (UC) is a complex disease characterized by chronic intestinal inflammation and is thought to be caused by an abnormal immune response to intestinal microbiota in genetically predisposed patients. (See e.g., C. Abraham et al., “Inflammatory Bowel Disease,” New England Journal of Medicine 361, 2066 (2009), which is incorporated herein by reference for all purposes). Treatment of UC may include aminosalicylates and steroids and, if low disease activity is not achieved, biologics such as tumor necrosis factor-α inhibitors (TNFi) may be recommended. (See e.g., S. C. Park et al., “Current and emerging biologics for ulcerative colitis,” Gut and liver 9, 18 (2015); K. Hazel et al., Emerging treatments for inflammatory bowel disease, “Therapeutic advances in chronic disease.” 11, 2040622319899297 (2020), which are incorporated herein by reference for all purposes). Nonetheless, about 40% of patients may be unresponsive to TNFi treatment, and up to 10% of initial responders may lose their response to TNFi therapy each year. (See e.g., S. C. Park et al.; P. Rutgeerts et al., “Infliximab for induction and maintenance therapy for ulcerative colitis,” New England Journal of Medicine 353, 2462 (2005), which are incorporated herein by reference for all purposes). Difficulties with TNFi therapies along with financial incentives led to research and development of alternative therapeutic approaches, for example, JAK inhibitors, IL-12/IL-23 inhibitors, SIP-receptor modulators, anti-integrin agents, or novel TNFi compounds. (See e.g., E. Troncone et al., “Novel therapeutic options for people with ulcerative colitis: an update on recent developments with Janus kinase (JAK) inhibitors,” Clinical and Experimental Gastroenterology 13, 131 (2020); A. Kashani et al., “The Expanding Role of Anti-IL-12 or Anti-IL-23 Antibodies in the Treatment of Inflammatory Bowel Disease,” Gastroenterology & Hepatology 15, 255 (2019); S. Danese et al., “Targeting S1P in inflammatory bowel disease: new avenues for modulating intestinal leukocyte migration,” Journal of Crohn's and Colitis 12, S678 (2018); S. C. Park et al., “Anti-integrin therapy for inflammatory bowel disease,” World journal of gastroenterology 24, 1868 (2018); K. Hazel et al., which are incorporated herein by reference for all purposes). Some approaches target biological mechanisms contributing to aberrant immune response and may require detailed knowledge about UC pathogenesis. However, due to concerns around immunogenicity and inconvenience of drug delivery through injections, there is an increasing interest in development of additional orally administered small molecule drugs.

Development of novel drugs may require identification of molecular targets whose modulation may lead to low disease activity or remission. With the surge in multi-omic data, machine learning (ML) and artificial intelligence (AI) became widely used for many tasks in therapeutics such as target prioritization, drug design, drug target interaction prediction, or small molecule optimization. (See e.g., J. Vamathevan et al., “Applications of machine learning in drug discovery and development,” Nature reviews Drug discovery 18, 463 (2019), which is incorporated herein by reference for all purposes). Current ML/AI approaches for target prioritization may focus on searching for genes involved in a given disease. Genes may be inferred by e.g., training classifiers using features constructed from a disease-specific gene expression and mutation data, along with information about relevant protein-protein, metabolic, or transcriptional interactions, or by analyzing existing textual databases or research literature for disease-genes associations using natural language processing (NLP) methods. (See e.g., P. R. Costa et al., in BMC Genomics, Vol. 11 (Springer, 2010) pp. 1-15; J. Jeon et al., “A systematic approach to identify novel cancer drug targets using machine learning, inhibitor design and high-throughput screening,” Genome medicine 6, 1 (2014); E. Ferrero et al., “In silico prediction of novel therapeutic targets using gene-disease association data,” Journal of translational medicine 15, 1 (2017); P. Mamoshina et al., “Machine learning on human muscle transcriptomic data for biomarker discovery and tissue-specific drug target identification,” Frontiers in genetics 9, 242 (2018); A. Bravo et al., “Extraction of relations between genes and diseases from text and large-scale data analysis: implications for translational research,” BMC Bioinformatics 16, 1 (2015); J. Kim et al., “An analysis of disease-gene relationship from Medline abstracts by DigSee,” Scientific Reports 7, 1 (2017), which are incorporated herein by reference for all purposes).

Yet, many ML/AI approaches may suffer from exploration biases or data incompleteness. (See e.g., T. Rolland et al., “A proteome-scale map of the human interactome network,” Cell 159, 1212 (2014); J. Menche et al., “Uncovering disease-disease relationships through the incomplete interactome,” Science 347, 1257601 (2015), which are incorporated herein by reference for all purposes). Moreover, systematic analyses demonstrated that drugs approved by the U.S. Food and Drug Administration (FDA) may not directly target protein products of the disease-associated genes. (See e.g., M. A. Y1ld1r1m et al., “Drug—target network,” Nature biotechnology 25, 1119 (2007); E. Guney et al., “Network-based in silico drug efficacy screening,” Nature communications 7, 1 (2016), which are incorporated herein by reference for all purposes). Network-based target prioritization methods may address these issues by aggregating proteomic, metabolomic, and transcriptomic interactions as well as associations between drugs, diseases, and genes in the form of networks and by deriving the network-based features distinguishing feasible targets in an unbiased and unsupervised manner. (See e.g., S. Zhao et al., “Network-based relating pharmacological and genomic spaces for drug target identification,” PloS one 5, e11764 (2010); Z. Isik et al., “Drug target prioritization by perturbed gene expression and network information,” Scientific reports 5, 1 (2015); T. Katsila et al., “Computational approaches in target identification and drug discovery,” Computational and structural biotechnology journal 14, 177 (2016); E. Guney et al., which are incorporated herein by reference for all purposes). Nonetheless, there is not yet a network-based framework that simultaneously captures the relation between disease formation and successful treatment as a method to identify novel potential targets.

To address at least these issues, disclosed herein are network-based methods for target prioritization for UC that utilizes three network regions (modules) of a Human Interactome (HI)—a network of protein-protein interactions in human cells—referred to as a module triad comprising:
1. Genotype module—a set of genes associated to the genetic predisposition of UC;
2. Response module—a set of genes whose expression needs to be altered in order to achieve low disease activity;
3. Treatment module—a set of proteins that need to be targeted to alter expression of Response module genes in a favorable direction to achieve low disease activity.

Feasible targets may simultaneously (a) be topologically relevant to the Genotype module, e.g., be in the network vicinity of the genes associated with a particular disease and (b) be functionally similar to the Treatment module, e.g., have a similar transcriptomic downstream effects to that of the Treatment module proteins upon their perturbation. (See e.g., E. Guney et al.). Methods disclosed herein may demonstrate the utility of the proposed framework, using UC as an example, by efficiently recovering known targets approved for UC and distinguishing targets being at different stages of development for UC based on network-derived rankings. The module triad framework may be the first attempt to connect biological mechanisms underlying complex disease development and its treatment dynamics from the network perspective. The module triad framework may be directly extendable to other complex diseases with known gene-disease associations, available gene expression data of patients before and after treatment, and perturbation experiments in appropriate cell lines.

### Overview of the Module Triad Target Prioritization Framework

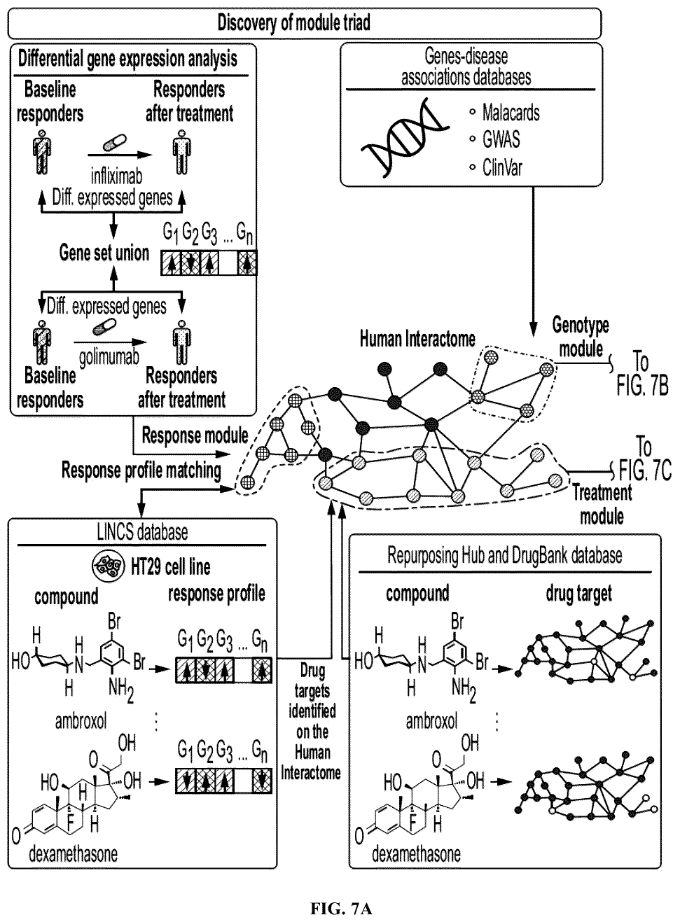

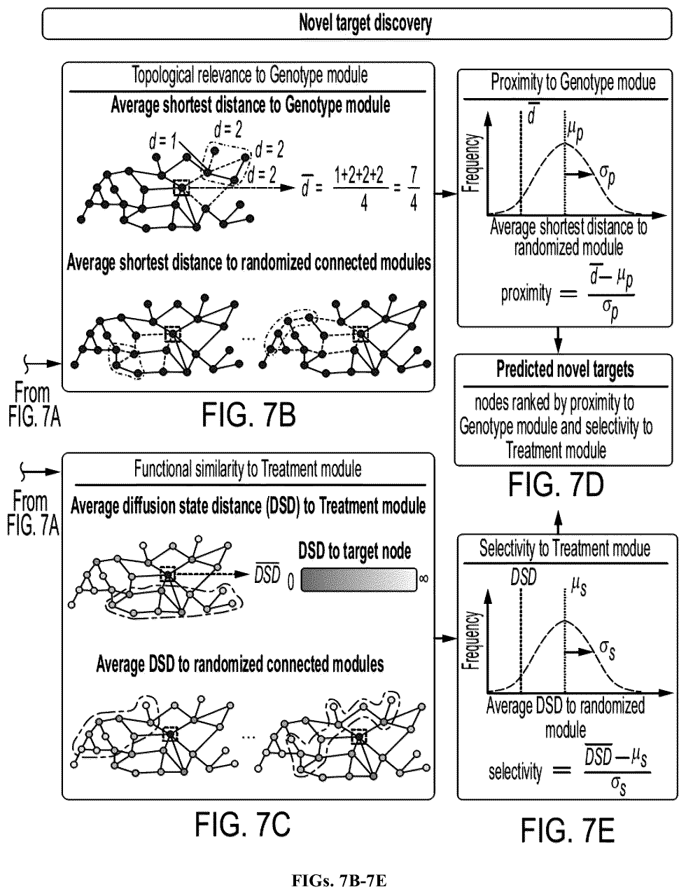

The module triad framework comprises: (1) discovery of the module triad for a given disease; (2) novel target discovery based on the identified module triad, which are illustrated in FIGS. 7A-7E.

For discovery of the module triad, each module may be mapped to the HI using auxiliary disease-specific information. The Genotype module may be constructed by analyzing gene-disease associations databases to locate genes whose mutations may predetermine the formation of the disease phenotype. The Response module comprises the genes that may be significantly down- or up-regulated after treatment in patients that achieved low disease activity. Treatment module construction comprises: (1) using the Library of Integrated Network-Based Cellular Signatures (LINCS) L1000 perturbations database to identify small molecule compounds that result in gene expression profiles similar to that observed for Response module genes after treatment; (2) using the DrugBank and Repurposing Hub databases to extract the set of proteins targeted by these compounds; these proteins are mapped to the HI resulting in the Treatment module. (See e.g., A. Subramanian et al., “A next generation connectivity map: L1000 platform and the first 1,000,000 profiles,” Cell 171, 1437 (2017); C. Knox et al., “DrugBank 3.0: a comprehensive resource for ‘omics’ research on drugs,” Nucleic acids research 39, D1035 (2010); S. M. Corsello et al., “The Drug Repurposing Hub: a next-generation drug library and information resource,” Nature medicine 23, 405 (2017), which are incorporated herein by reference for all purposes).

At least some proteins (nodes) of the HI are ranked based, at least in part, on the constructed Genotype and Treatment modules. For each node, its topological relevance to the Genotype module is assessed based on its proximity which is computed based on the average shortest distance from the node to the Genotype module nodes. (See e.g., E. Guney et al.). Functional similarity of the node to the Treatment module is assessed using selectivity which is computed based on the average diffusion state distance (DSD) of the node to the Treatment module nodes. (See e.g., M. Cao et al., “Going the distance for protein function prediction: a new distance metric for protein interaction networks,” PloS one 8, e76339 (2013), which is incorporated herein by reference for all purposes). For details on computing proximity and selectivity, see FIGS. 7A-7E and Methods (described elsewhere herein). HI nodes can be ranked based on their proximity and selectivity scores, and these two rankings can be merged into a single combined rank using the rank product. (See e.g., R. Breitling et al., “Rank products: a simple, yet powerful, new method to detect differentially regulated genes in replicated microarray experiments,” FEBS letters 573, 83 (2004), which is incorporated herein by reference for all purposes).

### UC Genotype Module

Protein products of genes associated with a disease usually are not randomly scattered on the HI but rather form clusters of interconnected nodes reflecting the existence of an underlying biological mechanism behind disease formation. (See e.g., J. Xu et al., Discovering disease-genes by topological features in human protein-protein interaction network,” Bioinformatics 22, 2800 (2006); K.-I. Goh et al., “The human disease network,” Proceedings of the National Academy of Sciences 104, 8685 (2007); T. Ideker et al., “Protein networks in disease,” Genome research 18, 644 (2008); A.-L. Barabási et al., “Network medicine: a network-based approach to human disease,” Nature reviews genetics 12, 56 (2011), which are incorporated herein by reference for all purposes). Studying network properties of these interconnected clusters has advanced understanding of disease molecular mechanisms, target discovery, and drug repurposing. (See e.g., J. Menche et al.; A. Sharma et al., “A disease module in the interactome explains disease heterogeneity, drug response and captures novel pathways and genes in asthma,” Human molecular genetics 24, 3005 (2015); E. Guney et al.; F. Cheng et al., “Network-based approach to prediction and population-based validation of in silico drug repurposing,” Nature communications 9, 1 (2018), which are incorporated herein by reference for all purposes).

To include the notion of UC genetic associations in the module triad framework, GWAS Catalog, ClinVar, or MalaCards databases may be used to extract genes reported to have associations with UC (see Methods described elsewhere herein). (See e.g., A. Buniello et al., “The NHGRI-EBI GWAS Catalog of published genome-wide association studies, targeted arrays and summary statistics 2019,” Nucleic acids research 47, D1005 (2019); M. J. Landrum et al., “Clin Var: improving access to variant interpretations and supporting evidence,” Nucleic acids research 46, D1062 (2018); N. Rappaport et al., “MalaCards: an integrated compendium for diseases and their annotation,” Database 2013 (2013), which are incorporated herein by reference for all purposes). A total of 194 genes were reported in at least one of the three databases as being associated with UC, and 174 of them (89.7%) are mapped to their corresponding protein products in the HI. The protein products are not randomly scattered on the network; 64.9% (113 174) of proteins are interconnected, forming a largest connected component (LCC) that is significantly larger than expected at random (e.g., Z-score=4.82, p<10⁻⁴). Methods described herein define this LCC as the Genotype module representing genetic predispositions to UC. A feasible target may be located in the topological vicinity of the Genotype module. (See e.g., E. Guney et al.).

### Successful UC Treatment is Reflected at the Transcriptomic Level

Besides being topologically close to the genes leading to predisposition to UC, a feasible target may also be functionally relevant to the treatment of UC. For example, UC treatment dynamics may be reflected at the transcriptomic level, and perturbing a feasible target may result in transcriptional changes similar to that observed upon successful UC treatment.

UC treatment may be reflected at the transcriptomic level in gene expression data of normal tissue controls and patients with active UC undergoing treatment with TNFi drugs, either infliximab or golimumab, from several studies. (See e.g., I. Arijs et al., “Mucosal gene expression of antimicrobial peptides in inflammatory bowel disease before and after first infliximab treatment,” PloS one 4, e7984 (2009); G. Toedter et al., “Gene expression profiling and response signatures associated with differential responses to infliximab treatment in ulcerative colitis,” Official journal of the American College of Gastroenterology—ACG 106, 1272 (2011); S. Pavlidis et al., “I MDS: an inflammatory bowel disease molecular activity score to classify patients with differing disease-driving pathways and therapeutic response to anti-TNF treatment,” PLOS Computational Biology 15, e1006951 (2019); N. Planell et al, “Transcriptional analysis of the intestinal mucosa of patients with ulcerative colitis in remission reveals lasting epithelial cell alterations,” Gut 62, 967 (2013); T. Montero-Melendez et al., “Identification of novel predictor classifiers for inflammatory bowel disease by gene expression profiling,” PloS one 8, e76235 (2013); J. T. Bjerrum et al., “Transcriptional analysis of left-sided colitis, pancolitis, and ulcerative colitis-associated dysplasia,” Inflammatory bowel diseases 20, 2340 (2014); S. E. Telesco, et al., “Gene expression signature for prediction of golimumab response in a phase 2a open-label trial of patients with ulcerative colitis,” Gastroenterology 155, 1008 (2018), which are incorporated herein by reference for all purposes). Table 4 summarizes TNFi treatment studies used to identify a molecular signature of UC patient response.

TABLE 4

| | | | | | | | |
|---|---|---|---|---|---|---|---|
| | | | | | | Pre- | Post- |
| GEO | | UC | Number of | TNFi | Response | treatment | treatment |
| accession | Normal | active | patients/normal | response | label | expression | expression |
| number | controls | patients | controls | label | timepoints | data | data |

Infliximab, Affymetrix ™ U133 Plus 2 microarray

| | | | | | | | |
|---|---|---|---|---|---|---|---|
| GSE16879 | + | + | 24/6 | + | week 4-6 | + | + |
| GSE23597 | − | + | 45/− | + | week 8, 30 | + | + |
| GSE38713 | + | + | 14/13 | − | − | + | − |
| GSE13367 | − | + | 8/− | − | − | + | − |
| GSE36807 | + | + | 15/7 | − | − | + | − |
| GSE47908 | + | + | 39/15 | − | − | + | − |

Golimumab, Affymetrix ™ U133 + microarray

| | | | | | | | |
|---|---|---|---|---|---|---|---|
| GSE92415 | + | + | 87/21 | + | week 6 | + | + |

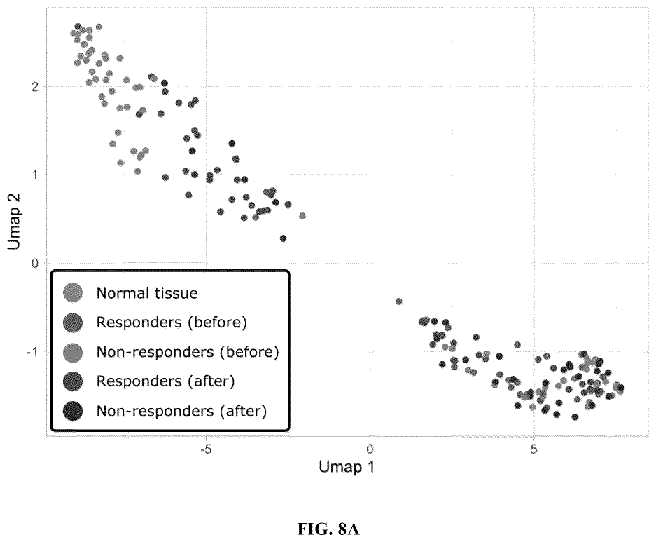

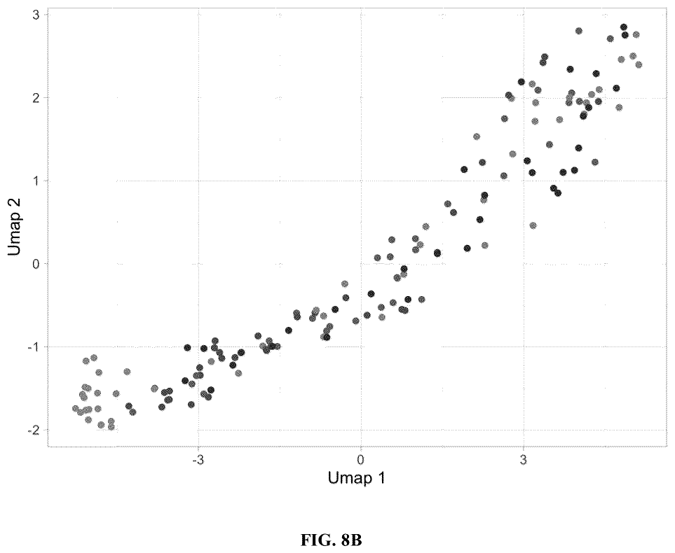

A set of 545 genes may be identified that are differentially expressed between patients with active UC and normal controls. These genes may be used as features for Uniform Manifold Approximation and Projection (UMAP) embedding of the gene expression profiles of normal controls and UC patients before and after treatment, split into two groups: patients who achieved low disease activity after treatment (responders) and those who did not (non-responders). (See FIGS. 8A-8B). (See e.g., L. McInnes et al., “Umap: Uniform manifold approximation and projection for dimension reduction,” arXiv preprint arXiv:1802.03426 (2018), which is incorporated herein by reference for all purposes).

From UMAP embedding, apparent distinction may not be observed between the pre-treatment gene expression profiles of responders and non-responders to infliximab or golimumab. Additionally, differentially expressed genes may not be found between the pre-treatment gene expression profiles of responders and non-responders. (See “differential gene expression analysis of responders and nonresponders to TNFi therapy,” described elsewhere herein). Conversely, the post-treatment gene expression profiles of responders are clustered closely with those of normal controls, whereas post-treatment profiles of non-responders to infliximab or golimumab are clustered separately from those of normal controls, indicating that gene expression profiles with high similarity to those of normal controls may be reflective of successful UC treatment. Motivated by these observations, we define “molecular response” to UC treatment as reversal of the gene expression profile of UC patients upon treatment to resemble the gene expression profiles of normal controls.

### UC Response Module

To further understand what transcriptional changes may cause responders' gene expression profile to become more similar to those of normal controls, differential expression analysis of pre- and post-treatment gene expression profiles of responders were performed. A small fraction of genes dysregulated in responders before treatment with respect to normal controls exhibits significant changes in expression after treatment (See “differential gene expression analysis of responders and nonresponders to TNFi therapy,” described elsewhere herein). Expression of these genes may be reverted in responders upon treatment e.g., genes down-regulated in responders before treatment with respect to normal controls may become up-regulated after treatment and vice versa. Yet, these transcriptional changes may be sufficient to make the gene expression profiles of responders and normal controls similar based on the profile embeddings shown in FIGS. 8A-8B and are indicative of patients who achieved low disease activity following treatment. This set of genes indicative of molecular response to UC treatment may be called the RBA (responders before-after) set. The RBA set specific to TNFi treatment of UC may be constructed by taking the union of RBA genes determined from the infliximab- and golimumab-based studies. (See Methods described elsewhere herein).

Genes belonging to the RBA set may be related to each other via one or multiple biological pathways, proper functioning of which may be restored by inhibition of TNF-α, and therefore may be located close to each other on the HI. To test this, TNFi RBA genes may be mapped on the HI to construct a subnetwork comprised of the nodes corresponding to the RBA genes. The RBA set forms a significant LCC on the HI (91 out of 271 nodes, 34%) as compared to a randomly selected set of nodes with preserved degree sequence (Z-score=9.24, p<10⁻⁴). This refined set of genes in the RBA LCC is defined as the Response module, e.g., the region of the HI transcriptionally altered when a UC patient achieves low disease activity in response to therapeutic intervention.

### UC Treatment Module

Successful treatment of UC may require reverting the expression profile of the Response module nodes by studying the gene expression profiles of UC patients undergoing TNFi therapies. Inhibition of TNF-α may not be the only way to achieve predetermined transcriptomic effects in the Response module genes, and perturbation of other proteins may achieve similar downstream effects.

Alternative perturbations that are experimentally validated may be analyzed to result in a molecular response similar to the one observed upon successful TNFi therapy. Differential gene expression effects (signatures) may result from perturbation of human cell lines with small molecule compounds obtained from the LINCS L1000 database. (See e.g., A. Subramanian et al., “A next generation connectivity map: L1000 platform and the first 1,000,000 profiles,” Cell 171, 1437 (2017), which is incorporated herein by reference for all purposes). Perturbation signatures may be derived from LINCS L1000 Level 5 data containing gene-wise Z-scores that indicate the magnitude and direction of change in gene expression for 14,513 compound experiments in the HT29 cell line (e.g., human colorectal adenocarcinoma cell line). Perturbation experiments in the HT29 cell line may be considered because of its relevance to UC-affected tissue (colon) and relatively wide coverage of small molecule compounds.

To find the compounds and corresponding target proteins that revert expression of the Response module genes, the LINCS L1000 experiments may be assessed by computing the Weighted Connectivity Score (WTCS) with respect to the up- and down-regulated genes in the Response module using gene-wise perturbation Z-scores for each HT29 cell line experiment. (See e.g., A. Subramanian et al., “A next generation connectivity map: L1000 platform and the first 1,000,000 profiles,” Cell 171, 1437 (2017), which is incorporated herein by reference for all purposes). To assess statistical significance of the WTCS for a given experiment, a randomization procedure may be employed assigning a pair of p-values, pᵤₚ and p_{down}, associated with the enrichment scores of the up- and downregulated genes. (See Methods described elsewhere herein). Compound experiments that have pᵤₚ≥0.05 and p_{down}≥0.05, and WTCS≥0 are excluded. This filtering ensures consideration of compounds that have a positive and significant therapeutic effect in terms of reverting the expression of Response module genes. Of 14,513 compound experiments conducted in the HT29 cell line, 68 experiments have a statistically significant WTCS, ranging from −0.642 to −0.480. 69 proteins appear as a target for at least one of the 25 unique compounds evaluated in these 68 experiments, according to DrugBank™ and Repurposing Hub™ databases. Two proteins may not be mapped to the HI (e.g., they have no known protein interaction partners), and 43 out of 67 remaining proteins (64%) form a LCC of significant size (Z-score=3.39, p<10⁻⁴). This LCC is called the Treatment module.

One of the targets belonging to the Treatment module is TNF-α. Moreover, by construction, targeting proteins belonging to the Treatment module may result in transcriptional changes within the Response module similar to those observed upon successful TNFi therapy. Hence, proteins belonging to the Treatment module may offer intervention opportunities for treating UC patients.

### Target Ranking

Besides potential intervention opportunities suggested directly from the Treatment module nodes, the Genotype and Treatment modules can be used to prioritize, in an unsupervised fashion, all nodes in the HI for their potential as a UC treatment target. A feasible target may simultaneously satisfy the following network properties. A feasible target may be topologically close to HI nodes associated with genetic predisposition to UC (Genotype module). Target prioritization based on the network proximity of nodes to disease modules is predictive of therapeutic effects of drugs with known targets across multiple diseases. (See e.g., E. Guney et al.). Therefore, to quantify topological relevance of a given HI node to the UC Genotype module, its proximity to the Genotype module may be calculated based on the average network shortest path of the node to the Genotype module (see Methods described elsewhere herein).

Also, targeting a feasible target may cause transcriptional changes similar to those observed upon successful UC treatment. The Treatment module defines a network region consisting of nodes that, upon perturbation, may result in desirable transcriptional changes in Response module genes. Therefore, proteins that are functionally similar to Treatment module proteins may also be promising targets. Yet, to find such targets, a methodology may quantify downstream transcriptional effect similarities of HI nodes based on network structure. For this, diffusion state distance (DSD), a metric based on network random walks designed to capture propagation-based topological similarities between each pair of nodes in the network, may be used because of its superior performance in predicting protein functional annotations. (See e.g., M. Cao et al.).

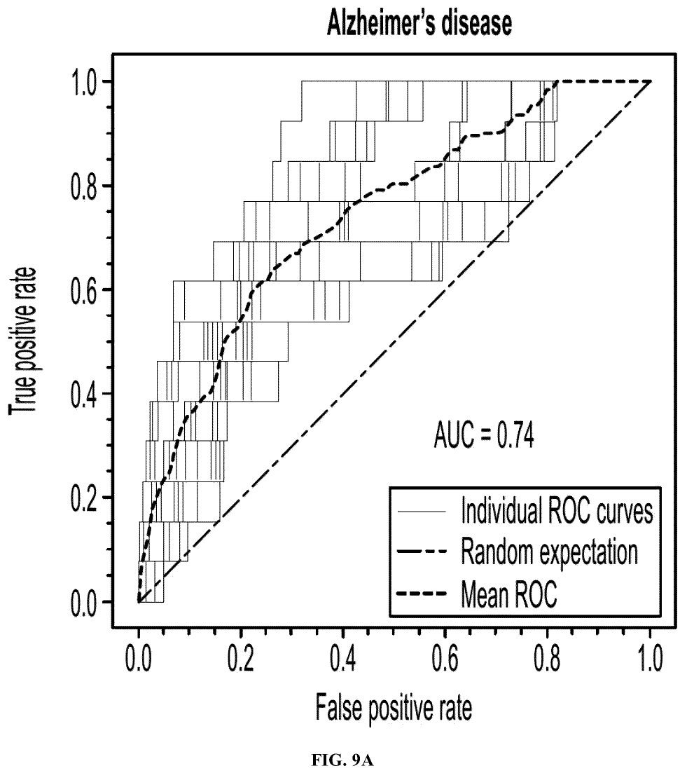

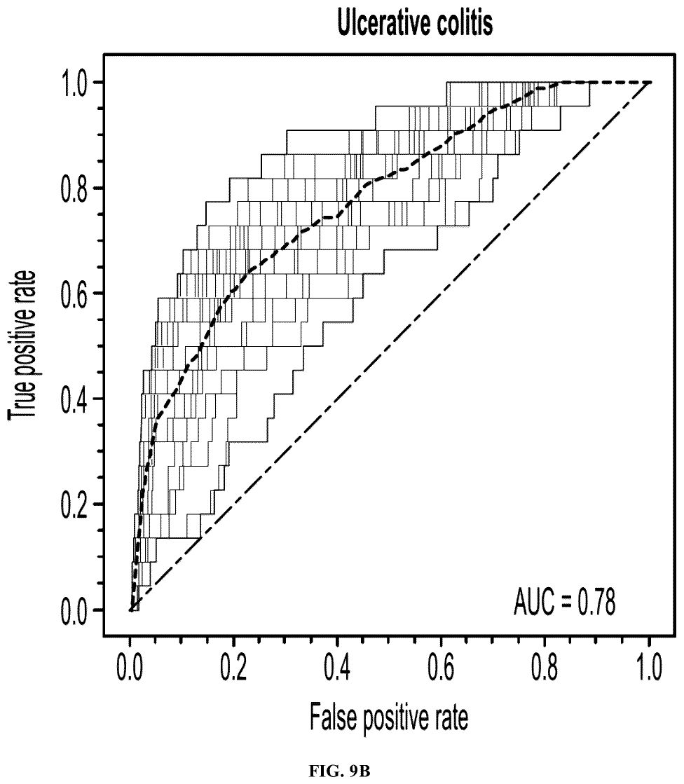

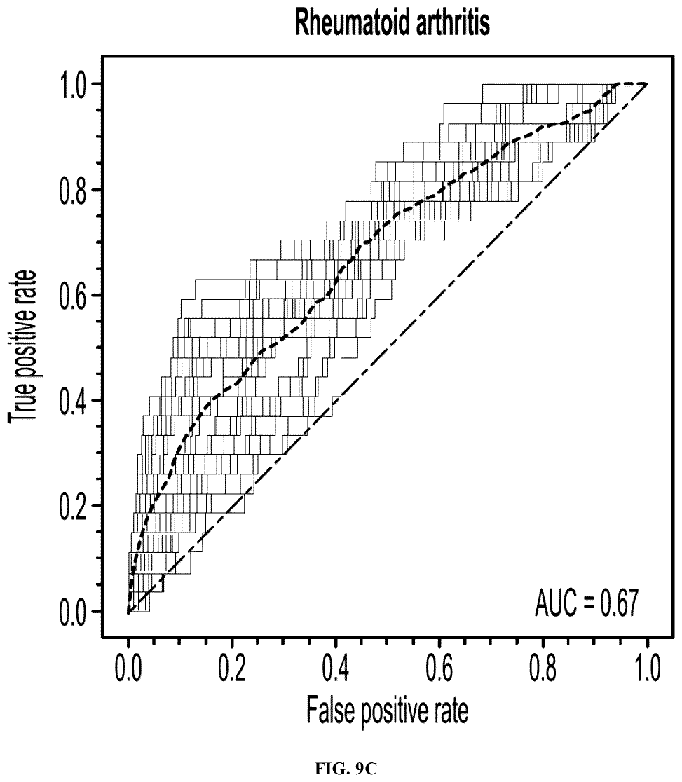

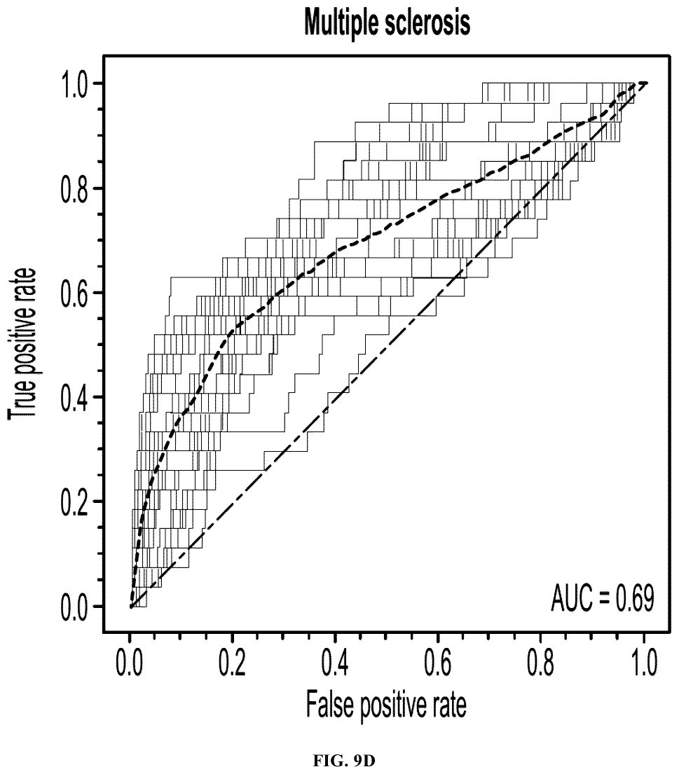

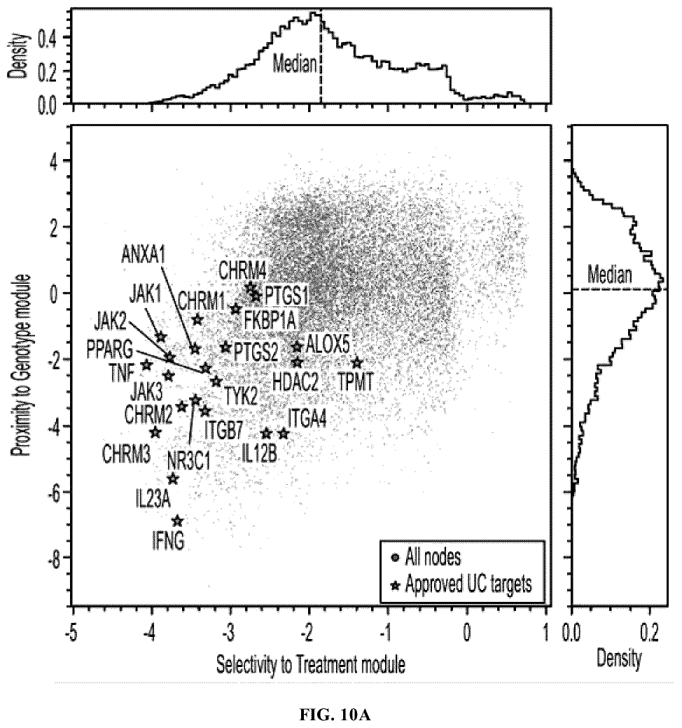

To evaluate whether DSD reflects similarities in downstream transcriptional effects between different proteins, the recovery of approved drugs for four complex diseases may be analyzed (e.g., Alzheimer's disease, ulcerative colitis, rheumatoid arthritis, and multiple sclerosis) based on DSD between the HI nodes. (See Methods, described elsewhere herein). The targets of each approved drug may result in similar therapeutic effects of treating a given disease. Thus, efficiently recovering approved targets may be possible by knowing one drug target and its DSD to other HI nodes. Such target recovery may be performed separately for each approved target and complex disease to derive receiver operator characteristic (ROC) curves as shown in FIGS. 9A-9D. Knowing DSD from an approved drug target to the rest of the nodes in the HI may be sufficient to recover the rest of the known approved targets in each complex disease.

Yet, a node that has low DSD to the Treatment module may be equally close to other randomly chosen modules of equal size in the HI. To account for this, functional similarity between HI nodes and the Treatment module may be quantified using selectivity e.g., a network-based measure based on the DSD that considers statistical significance of the DSD between a node and a given network module. (See Methods described elsewhere herein).

Finally, all HI nodes may be ranked based on their proximity to the Genotype module and selectivity to the Treatment module, and the rank product may be used to determine the final combined ranking of the nodes. (See Methods described elsewhere herein). (See e.g., R. Breitling et al.).

### In Silico Validation of the Module Triad Target Prioritization

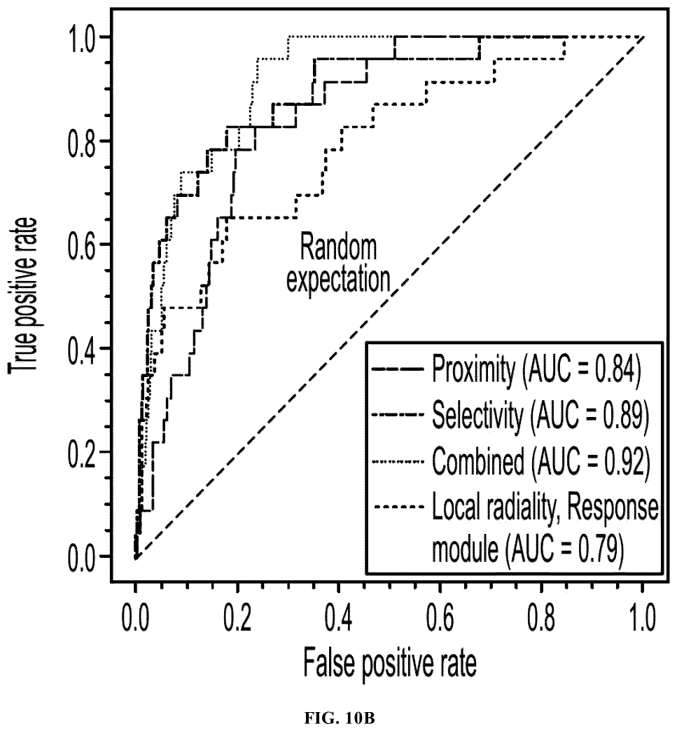

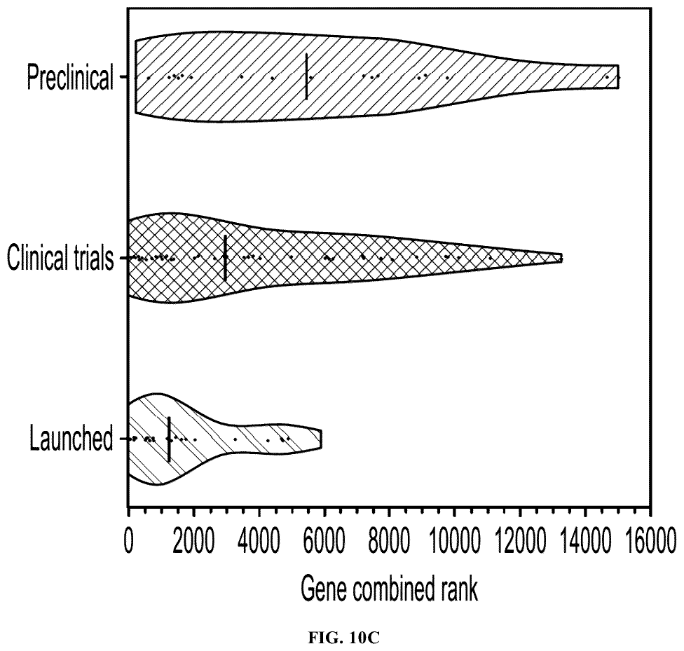

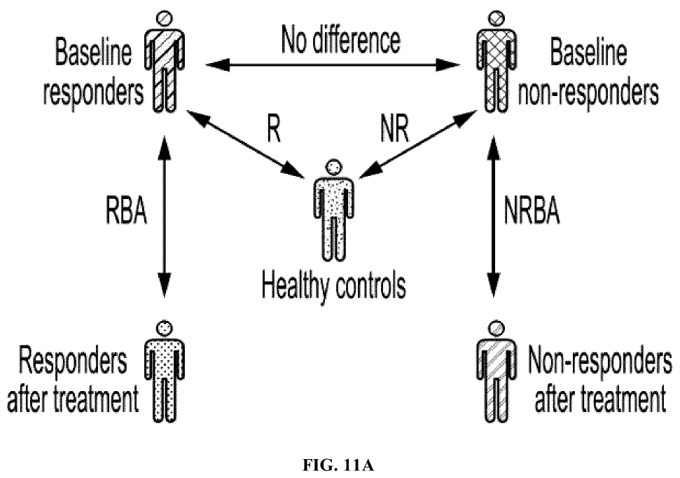

To test if the proposed target ranking yields meaningful results, drug targets approved for UC treatment were obtained from the PharmaIntelligence™ Citeline database. (See Methods described elsewhere herein). The resulting list comprises 23 targets mapped on the HI. The approved targets are simultaneously highly proximal to the Genotype module and selective to the Treatment module compared to the rest of HI nodes as shown in FIG. 10A. While both proximity and selectivity efficiently recover known approved targets on their own, a combination of both performs better suggesting a synergistic effect of these network measures for target prioritization as shown in FIG. 10B. In addition to the proposed network measures for target prioritization, another measure based on the combination of network and gene expression data, Local radiality, that has shown high performance in recovering known drug targets may be checked. (See e.g., Z. Isik et al.). Local radiality is similar to the module triad prioritization methods described herein, in that it employs both topological and gene expression data to prioritize targets. The main difference is that Local radiality assumes that HI nodes affected by perturbation of a target (downstream nodes) may be in the network vicinity of the target. Using methods described herein, targets can be prioritized based on their Local radiality with respect to the Response module nodes that reflect the predetermined downstream effect. (See Methods described elsewhere herein). Local radiality may also efficiently recover approved UC targets, albeit less efficiently than the module triad prioritization methods described herein. Sensitivities corresponding to approved UC target recovery for all tested methods are reported in Table 5 which shows fraction of recovered approved targets for UC treatment among top-K proteins ranked by selectivity, proximity, combined proximity and selectivity, and local radiality to the Response module.

TABLE 5

| Top-K ranked | Selectivity | Proximity | Combined | Local radiality |
|---|---|---|---|---|
| proteins | ranking | ranking | ranking | ranking |
| 10 | 0/23 | 0/23 | 0/23 | 0/23 |
| 50 | 2/23 | 1/23 | 1/23 | 1/23 |
| 100 | 3/23 | 1/23 | 3/23 | 1/23 |
| 500 | 11/23 | 2/23 | 8/23 | 8/23 |
| 1,000 | 14/23 | 5/23 | 12/23 | 10/23 |
| 5,000 | 19/23 | 19/23 | 22/23 | 15/23 |
| 10,000 | 22/23 | 23/23 | 23/23 | 20/23 |

Finally, drugs that are under consideration as a UC treatment (e.g., being tested in clinical and preclinical trials) may target nodes that have a lower combined ranking based on the proximity and selectivity when compared to the targets that are already launched for UC. This is because launched targets have already been assessed through clinical stages for their ability to ameliorate disease activity in UC patients, while targets that are not yet launched may not necessarily be efficacious for treatment of UC. Distribution of the combined ranks may be compared for the targets of drugs that are launched, in clinical trials (Phase I, II, III), or preclinical studies as shown in FIG. 10C. Median combined ranking of the targets corresponding to the launched drugs is higher, followed by those in clinical trials, followed by those in preclinical studies.

### Discussion

Described herein are a network-based framework and methods for prioritizing protein targets as novel therapies for complex diseases using UC as an example disease. The module triad framework is the first attempt at capturing both formation and successful treatment of disease at the network level assuming that the mechanism behind complex disease formation and treatment can be captured by the interplay between the three network modules of genetic predisposition, transcriptional changes, and protein targets of drugs on the HI. In methods described herein, formation of the disease phenotype is predetermined by the genetic mutations in a collection of genes that are localized in the HI region called the Genotype module. These genetic alterations within the Genotype module manifested in gene expression changes in patients with active UC. By tracking the genes whose expression levels changed significantly in the patients that achieved low disease activity upon TNFi therapy, a collection of genes may be derived that may be transcriptionally altered in order to achieve a positive response to the treatment. These genes occupy a localized region of the HI termed the Response module.

Proteins targeting may be identified which results in a similar transcriptional perturbation profile as achieved upon successful TNFi therapy. Methods described herein may do so by scanning the experimental data of the small molecule compounds perturbing human cells and matching the response profiles after compound perturbation with the profile achieved upon successful treatment. The collection of compound targets that achieve the predetermined downstream change of gene expression also occupies a localized region in the HI and is called the Treatment module. While the identified compounds matching the predetermined transcriptomic downstream effect may seem different, as illustrated in Table 6 (which indicates drugs and their known mechanisms of action mapped to the protein targets belonging to the Treatment module), their targets belong to a localized region of the HI, reflecting common underlying biology behind treatment of UC, and suggesting that other protein targets that are functionally similar to the Treatment module nodes are promising targets for UC treatment. By ranking the HI nodes based on their proximity to the Genotype module and selectivity to the Treatment module, methods disclosed herein may prioritize the HI proteins that are simultaneously topologically relevant to the genes associated with formation of UC phenotype and functionally similar to proteins that have desirable treatment downstream effect when being targeted

TABLE 6

| Drug name | Known mechanism of action |
|---|---|
| diethylstilbestrol | estrogen receptor agonist |
| dexamethasone- | glucocorticoid receptor agonist |
| acetate | |
| acarbose | glucosidase inhibitor |
| betaxolol | adrenergic receptor antagonist |
| avicin-d | AMP-activated protein kinase activation |
| piceatannol | SYK inhibitor |
| calcifediol | vitamin D receptor agonist |
| UNC-0321 | G9a inhibitor |
| homatropine | acetylcholine receptor antagonist |
| PD-184352 | MEK inhibitor |
| wortmannin | PI3K inhibitor |
| ERK-inhibitor-11E | ERK inhibitor |
| reversine | Aurora kinase inhibitor |
| vemurafenib | RAF inhibitor |
| PLX-4720 | RAF inhibitor |
| carbamazepine | carboxamide antiepileptic |
| leucodin | TNF-alpha, TIMP Metallopeptidase Inhibitor |

Proximity used for quantifying topological relevance of targets to Genotype module was shown to offer an unbiased measure of therapeutic effects across various drugs and diseases and for distinguishing palliative treatments from effective treatments. (See e.g., E. Guney et al.). Drugs whose targets are proximal to genes associated with a disease may be more likely to be effective than more distant drugs. (See e.g., E. Guney et al.). Methods described herein used DSD as a proxy for measuring similarity between downstream effects resulting from perturbing a given pair of nodes in the HI. DSD between a pair of nodes is based on similarity between random walks starting from these nodes. Visiting frequencies of random walkers per node were successfully used to assess perturbation patterns resulting from elementary mutations in genes related to cancer (e.g., single-nucleotide variations and insertion/deletion mutations). (See e.g., M. D. Leiserson et al., “Pan-cancer network analysis identifies combinations of rare somatic mutations across pathways and protein complexes,” Nature genetics 47, 106 (2015), which is incorporated herein by reference for all purposes). Visiting frequencies of the random walk starting from a given node may correspond to the amount of perturbation this node imposes on the rest of the network, and the downstream perturbation effect is reflected in the vector of visiting frequencies of the random walk starting at a given node. Since DSD measures the distance between the vectors of random walks' visiting frequencies (see Methods described elsewhere herein), a pair of nodes with small DSD corresponds to the nodes with similar downstream perturbation effects. DSD is indeed reflective of similarities between therapeutic effects of different targets by recovering known approved targets for 4 complex diseases, including UC, based on the DSD.

The module triad framework and methods disclosed herein may utilize knowledge about the treatment dynamics of patients with active UC that achieved low disease activity upon TNFi therapy. However, patients that do not demonstrate sufficient response to TNFi therapy represent a large fraction of diseased population and may potentially suffer from UC subtype that is different in its underlying biology or disrupts normal cellular processes more severely. (See “pathway enrichment analysis of differentially expressed genes in responders and non-responders to TNFi therapy,” described elsewhere herein). (See e.g., P. Rutgeerts et al.). While novel targets identified using methods described herein may help to find therapies suitable for TNFi non-responders, research of exact biology behind insufficient response to TNFi therapies may still be required.

The module triad framework and methods described herein utilizing patients genomic and transcriptomic data may offer a holistic network-based view on the formation and treatment dynamics of complex diseases and may provide an unbiased approach to novel target identification. Methods disclosed herein can be generalized to any complex disease with available gene-disease associations data, transcriptomic data of patients before and after treatment, and perturbation experiments in an appropriate cell line. Besides target prioritization, methods disclosed herein can suggest repurposing opportunities based on the targets belonging to the Treatment module. Module triad methods may be enhanced by considering available perturbation experiments such as single-gene overexpression and knockdown, including information about agonist or antagonist action of drugs on their targets, or by further refining the list of prioritized targets considering their toxicity and druggability.

### Methods

Human interactome. The HI map of experimentally derived protein-protein interactions is assembled from public databases. (See e.g., T. Mellors et al., “Clinical validation of a blood-based predictive test for stratification of response to tumor necrosis factor inhibitor therapies in rheumatoid arthritis patients,” Network and Systems Medicine 3, 91 (2020), which is incorporated herein by reference for all purposes). The HI used herein is assembled using e.g., database versions as of March 2021.

Construction of the UC Genotype module. Genes associated with UC are identified as indicated by the (1) GWAS catalog; (2) Clin Var database, specifically, genes that are indicated as “pathogenic”, “likely pathogenic”, and with “conflicting interpretations” of pathogenicity; and (3) MalaCards database. (See e.g., A. Buniello et al.; M. J. Landrum et al.; N. Rappaport et al.) The genes are collected from e.g., the databases as of September 2021. All the genes that are mentioned in at least one of the three databases may be retained, and the genes that are not part of the HI network may be filtered out. The remaining genes may be used to construct a subnetwork and to extract the largest connected component (LCC) of it.

Significance of the LCC size may be assessed by randomly sampling subnetworks with the degree sequence as in the original subnetwork. By repeatedly sampling 10,000 subnetworks, an empirical distribution may be found of the LCC size of randomly sampled subnetworks with its mean μ_{LCC} and standard deviation σ_{LCC}. Methods disclosed herein define the LCC Z-score as:

$${Z}_{L C C} = \frac{{S}_{L C C} - {μ}_{L C C}}{{σ}_{L C C}}$$

where S_{LCC} is the LCC size of the original subnetwork. Method disclosed herein also define the empirical p-value for the observed S_{LCC} as the fraction of the randomly sampled subnetworks that had their LCC size exceeding S_{LCC}.

Gene expression data processing for active UC cases and normal controls. Tissue mucosal samples were collected from normal controls and patients with moderately to severely active UC from Gene Expression Omnibus (GEO), as shown in Table 4. (See e.g., T. Barrett et al., “NCBI GEO: archive for functional genomics data sets—update,” Nucleic acids research 41, D991 (2012), which is incorporated herein by reference for all purposes). Three studies reported patient response statuses after treatment, where responses are determined by endoscopic and histologic findings or Mayo scores. See Table 7 for details on the response definition, for example, definitions of TNFi response across cohorts with specified UC patients' response labels. Methods disclosed herein obtained normalized data within each study from e.g., Gene Vestigator® database. (See e.g., T. Hruz et al., “Genevestigator v3: a reference expression database for the meta-analysis of transcriptomes,” Advances in bioinformatics 2008 (2008), which is incorporated herein by reference for all purposes).

TABLE 7

| GEO accession | |
|---|---|
| number | Definition of TNFi response |
| GSE16879 | “For UC and CDc, the response to infliximab was defined as a complete mucosal healing with |
| | a decrease of at least 3 points on the histological score for CDc and as a decrease to a Mayo |
| | endoscopic subscore of 0 or 1 with a decrease to grade 0 or 1 on the histological score for UC. |
| | (See e.g., S. C. Park et al.; M. Cao et al.; R. Breitling et al.) Patients who did not achieve this |
| | healing were considered nonresponders although some of them presented endoscopic or |
| | histologic improvement.” (See e.g., I. Arijs et al.) |
| GSE23597 | “ . . . defined as a decrease from baseline in the total Mayo score of at least three points and at |
| | least 30%, with an accompanying decrease in the subscore for rectal bleeding of at least one |
| | point or an absolute subscore for rectal bleeding of 0 or 1.” (See e.g., P. Rutgeerts et al.; G. |
| | Toedter et al.) |
| GSE92415 | “Response was defined as complete mucosal healing and histologic normalization (a Mayo |
| | endoscopic subscore of 0 or 1 and a grade of 0 or 1 on the Geboes histological scale).” (See |
| | e.g., S. E. Telesco et al.) |

Methods disclosed herein may integrate the expression data from 6 infliximab studies together. Batch effects among different studies are corrected using ComBat© statistical methods. (See e.g., J. T. Leek et al., “sva: Surrogate Variable Analysis R package version 3.10.0,” DOI 10, B9 (2014), which is incorporated herein by reference for all purposes). Some studies include baseline samples and samples collected at follow-up visits. To avoid underestimating variance introduced by analysis of longitudinal correlated samples, methods disclosed herein may apply ComBat© statistical methods to baseline samples to derive correction factors for individual studies, treating response and health status as covariates. The correction factors are implemented on baseline and follow-up visit samples.

Clustering and differential gene expression analysis. To reduce dimensionality of the gene expression data, methods disclosed herein may select a subset of gene features that are significantly differentially expressed between normal controls and UC active samples. Genes with fold change (FC) of FC>2.5 and adjusted p-value (Benjamini-Hochberg correction) of P_{adj.}<0.05 may be extracted. (See e.g., Y. Benjamini et al., “Controlling the false discovery rate: a practical and powerful approach to multiple testing,” Journal of the Royal statistical society: series B (Methodological) 57, 289 (1995), which is incorporated herein by reference for all purposes). For clustering analysis, methods disclosed herein may embed gene expression vectors of the identified differentially expressed genes into 8-dimensional space using UMAP. (See e.g., L. McInnes et al.).

When comparing the pre- and post-treatment gene expression profiles of the active UC patients, FC>1.8 and p_{adj.}<0.05 thresholds may be used to identify differentially expressed genes. The differentially expressed genes with negative log-fold change are considered significantly down-regulated while genes with positive log-fold change are considered significantly up-regulated. For more details on the paired analysis of differentially expressed genes, see “differential gene expression analysis of responders and nonresponders to TNFi therapy,” described elsewhere herein.

Construction of the UC Response module. To identify genes indicative of response to TNFi therapy, methods disclosed herein may extract the genes that are significantly differentially expressed in responders to infliximab and golimumab comparing their gene expression profiles before and after treatment as described above. The two RBA gene sets may be obtained from infliximab- and golimumab-based studies (see “differential gene expression analysis of responders and nonresponders to TNFi therapy,” described elsewhere herein), and a union of these two sets may be used to account for possible drug-specific gene expression changes. A subnetwork based on the obtained merged RBA gene set and the HI may be constructed. The LCC of the resulting subnetwork may be identified as the UC Response module and significance of its size analogously to the Genotype module may be assessed.

Analysis of LINCS 1.1000 perturbation profiles. Methods disclosed herein may assess the concordance between the differential gene expression profile upon perturbation of HT29 cells using various compounds and the genes belonging to the Response module split into up- and down-regulated subsets using Weighted Connectivity Score (WTCS). (See e.g., A. Subramanian et al., “A next generation connectivity map: L1000 platform and the first 1,000,000 profiles,” Cell 171, 1437 (2017), which is incorporated herein by reference for all purposes). WTCS measures the enrichment score, ES, of ranked lists of genes with a given pair of up- and down-regulated gene sets, that are referred to here as up- and down-query. (See e.g., A. Subramanian et al., “Gene set enrichment analysis: a knowledge-based approach for interpreting genome-wide expression profiles,” Proceedings of the National Academy of Sciences 102, 15545 (2005), which is incorporated herein by reference for all purposes, which is incorporated herein by reference for all purposes). WTCS combines the ES for up-query (ESᵤₚ) and down-query (ES_{down}) into a single score. A positive WTCS indicates that a perturbation resulted in a gene expression change that aligns with the Response module query set, e.g., up-query genes are also mainly up-regulated in a given perturbation while down-query genes are mainly down-regulated in a given perturbation. Conversely, a negative WTCS indicated that down-query genes are up-regulated in a given experiment while up-query genes are down-regulated. As we are interested in reverting expression patterns of the Response module genes, we look for experiments with negative WTCS. Below is the brief outline of the procedure used to compute this score and to assess its statistical significance.

LINCS L1000 Level 5 data stores differential gene expression profiles in terms of gene-specific Z-scores indicating changes in expression levels of genes with respect to controls. Large positive Z-score indicates that a gene is significantly up-regulated upon perturbation, while large negative Z-score indicates that a gene is significantly down-regulated upon perturbation. Genes for which differential expression patterns are inferred with high fidelity belong to the set of Best INferred Genes (BING) and are used for WTCS computation. (See e.g., A. Subramanian et al., “A next generation connectivity map: L1000 platform and the first 1,000,000 profiles,” Cell 171, 1437 (2017), which is incorporated herein by reference for all purposes). Up-regulated and down-regulated genes observed in the Response module that are also part of the BING set are denoted here as sᵤₚ and s_{down}, respectively. For each sets, methods disclosed herein may calculate enrichment scores (ESᵤₚ and ES_{down}), and WTCS is a combination of these two scores:

$$W T C S = \{ \begin{matrix}\frac{1}{2} ( E {S}_{u p} - E {S}_{d o w n} ) , & if sign ( {ES}_{u p} ) \neq sign ( {ES}_{d o w n} ) \\ 0 , & otherwise\end{matrix} .$$

To assess the significance of the enrichment scores, genes sets of sizes |sᵤₚ|, |s_{down}| may be sampled uniformly from BING genes. By repeating the sampling procedure 1,000 times, empirical distributions of up- and down-enrichment scores from random samples, ρᵤₚ(ES), ρ_{down} (ES), may be obtained. The obtained distributions may be compared to the observed ESᵤₚ and ES_{down}: if the observed ESᵤₚ is positive, the fraction of random samples which has greater or equal enrichment scores is selected as the p-value pᵤₚ, and if it is negative, the fraction of random samples which has smaller or equal enrichment scores is selected as the p-value pᵤₚ. The p_{down} is computed in a similar fashion. WTCS, pᵤₚ, and p_{down} may be obtained for each perturbation experiment and use them for filtering the relevant perturbations.

Construction of UC Treatment module. Using LINCS L1000 data, methods disclosed herein may identify compounds that are able to revert the expression patterns observed in the Response module nodes. Relevant experiments may be extracted using WTCS<0 and pᵤₚ<0.05, p_{down}>0.05 filters described above. The protein targets of the compounds remained after the filtering are identified using DrugBank and Repurposing Hub databases. We then map the resulting set of protein targets on the HI, and construct a subnetwork based on it analogously to the construction of the Response and Genotype modules. Treatment module is the LCC of this subnetwork.

Diffusion state distance. Diffusion state distance (DSD) is a metric defined on network nodes originally designed to predict proteins' functions in protein interaction networks. (See e.g., M. Cao et al.) DSD captures similarities between network's final states when random walkers start from two different nodes. To define the DSD, we first define He(vᵢ,vⱼ)—an expected number of times a random walk (RW) starting at node vᵢ and proceeding for k operations may end up at node vⱼ. Next, for node vᵢ, we define a vector

$$He ( {v}_{i} ) = \{ H e ( {v}_{i} , {v}_{1} ) , … , He ( {v}_{j} , {v}_{n} ) \} .$$

Then the DSD between nodes vᵢ and vⱼ is defined as

$$D S D ( {v}_{i} , {v}_{j} ) = {‖ He ( {v}_{i} ) - He ( {v}_{j} ) ‖}_{1} ,$$

where ∥ . . . ∥₁ denotes the L₁ norm. For any fixed k, DSD is a metric and it converges as k→∞. (See e.g., M. Cao et al.).

DSD as a measure of therapeutic similarity between targeted proteins. To quantify relevance of DSD as a measure of therapeutic effect similarity between proteins, a set of complex diseases and their approved targets may be analyzed through: for each of the known approved targets for a given disease, compute DSDs between that target and the rest of the nodes in the HI; rank the rest of the nodes based on the DSD to a known target, and based on that ranking, construct a receiver operator characteristic (ROC) curve corresponding to the recovery of the rest of the approved targets for a given disease. By iterating over all known approved targets, a set of individual ROC curves is obtained for each of complex diseases. Interpolation may be used to average the individual curves and to obtain the mean ROC curve, and compute the area under it, quantifying the likelihood of finding approved targets given knowledge about a single approved target and its DSD to the rest of the network nodes.

Proximity to UC Genotype module. Computing proximity of a node to the Genotype module comprises. computing the average shortest path length d̅from a given node to the nodes of the Genotype module; assessing the statistical significance of the closeness of the node to the Genotype module by comparing the average shortest path length to the Genotype module to the average shortest path distance to randomized network modules of the same size. Specifically, methods disclosed herein sample connected modules of the same size as the Genotype module (see below for sampling details) 500 times and construct an empirical distribution of the average shortest path distances to the randomized modules, with μₚ being the mean, and σₚ being the standard deviation of this distribution. Finally, proximity of the node is defined as the Z-score of the average shortest path distance from the node to the Genotype module with respect to this distribution:

$$proximity = \frac{\overline{d} - {μ}_{p}}{{σ}_{p}}$$

Selectivity to UC Treatment module. Computing selectivity of a node to the Treatment module is similar to computation of proximity comprising: computing the average DSD (D̅S̅D̅) of a node with respect to the nodes of the Treatment module; assessing statistical significance of the observed DSD by sampling 500 randomized network modules of the same size as the Treatment module, analogously to the proximity calculation. However, instead of the average shortest path distance, we compute the average DSD of the node to each randomized module and construct an empirical distribution of the average DSDs to the randomized modules, with μₛ being the mean and σₛ being the standard deviation of this distribution. We define selectivity as:

$$selectivity = \frac{\overline{D S D} - {μ}_{s}}{{σ}_{s}}$$

Network module randomization. Both proximity and selectivity computations may require sampling of randomized modules on the HI. As by construction both Genotype and Treatment modules are connected subnetworks, sampling connected subnetworks uniformly from the fixed HI network may avoid any possible biases of the average shortest path length or DSD with respect to the subnetwork connectedness. Neighbor Reservoir Sampling (NRS) algorithm may be used to sample connected fixed-size subnetworks uniformly. (See e.g., X. Lu et al., “International Conference on Scientific and Statistical Database Management,” Springer, (2012) pp. 195-212, which is incorporated herein by reference for all purposes).

Node ranking based on proximity and selectivity. Given the Genotype and Treatment modules, we compute proximity and selectivity scores of all nodes in the HI, and derive their corresponding ranks, rₚ and rₛ, respectively. To obtain a single combined rank r for each node, we used the rank product defined as:

$$r = \sqrt{{r}_{p} \cdot {r}_{s}}$$

Local radiality with respect to the Response module. Local radiliaty of node i with respect to the Response module may be determined using the following equation:

$$L {R}_{i} = \frac{{Σ}_{g ϵ RM} s p l ( i , g , G )}{\vert R M \vert}$$

where RM is the set of the Response module nodes, G is the Human Interactome network, spl(i,g,G) is the function measuring the length of the shortest path from node i to node g.

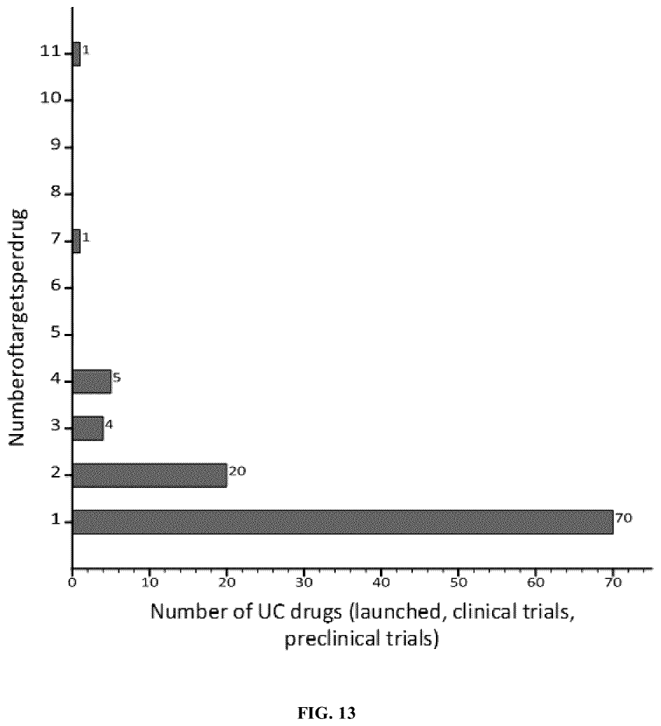

UC approved targets. For validation of the proposed target prioritization framework, a list of targets that are approved for UC treatment may be compiled by retrieving a list of all drugs with a status of launched or in development for UC using e.g., the PharmaIntelligence™ Citeline database as of February 2022. All drugs that are launched for UC are considered as approved drugs. Additionally, drugs are considered that are being tested for UC in clinical trials (Phase I, II, and III) and preclinical trials to compare their combined rankings to those of the approved drugs. For each drug, extract its known targets from e.g., the Pharma Intelligence™ Citeline database, Repurposing Hub database, and DrugBank database. Since a target may be mapped to several drugs, assign the highest reached status to a target based on the statuses of the drugs it is mapped to. For example, if a target is mapped to the two drugs, one of which is in Phase II clinical trials, and one of which is in preclinical trials, the target is labelled as the clinical trials target. Moreover, to avoid drugs that may have potentially many off targets due to high drug promiscuity, filter out the two drugs (sulfasalazine and mesalazine) that have more than 4 targets as shown in FIG. 13. (See e.g., V. J. Haupt et al., “Drug promiscuity in PDB: protein binding site similarity is key,” PLOS one 8, e65894 (2013), which is incorporated herein by reference for all purposes). Besides these two drugs, all other drugs being developed for UC treatment have 4 or less targets simultaneously. Additionally, filter out tetracosactide due to ambiguous indications for UC.

### Further Description of the Module Triad

Differential gene expression analysis of responders and nonresponders to TNFi therapy. To assess if responders and non-responders to TNFi therapies can be stratified based on gene expression profiles before treatment, methods disclosed herein may perform differential gene expression analysis using their full gene expression profiles. Significant differences may not be found at the fold change (FC) of FC=1.8 and adjusted p-value (Benjamini-Hochberg correction) of p<0.05. Therefore, evident differences may not exist between responders' and non-responders' before treatment neither in the UMAP embedding space, nor in the actual full gene expression profile space.

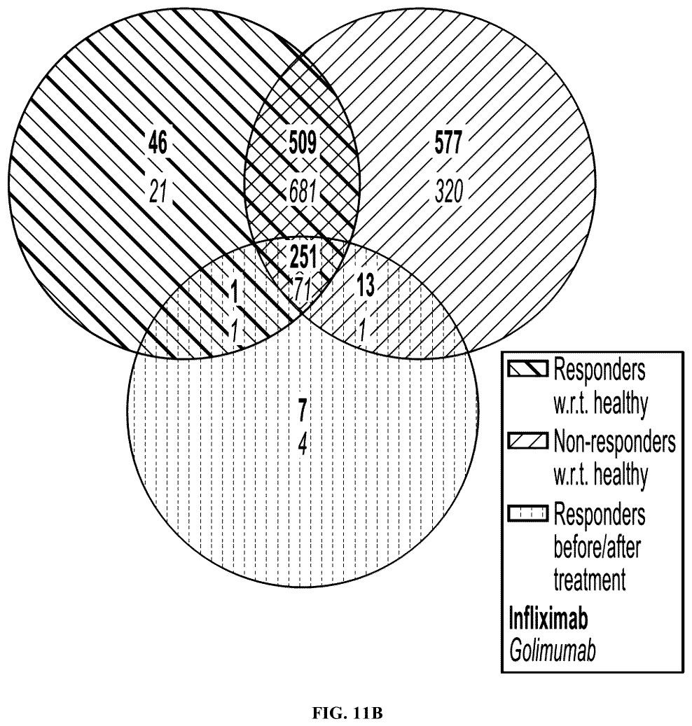

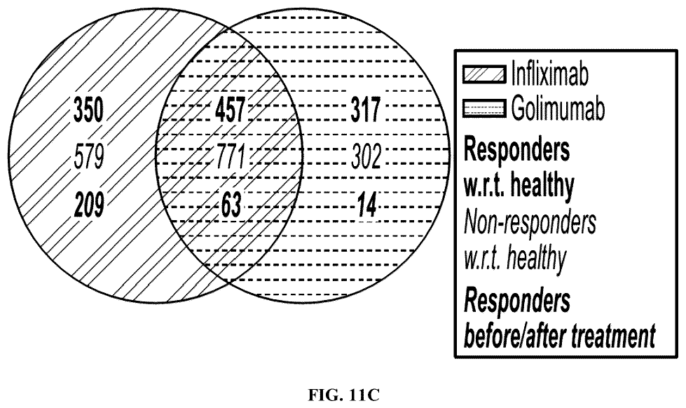

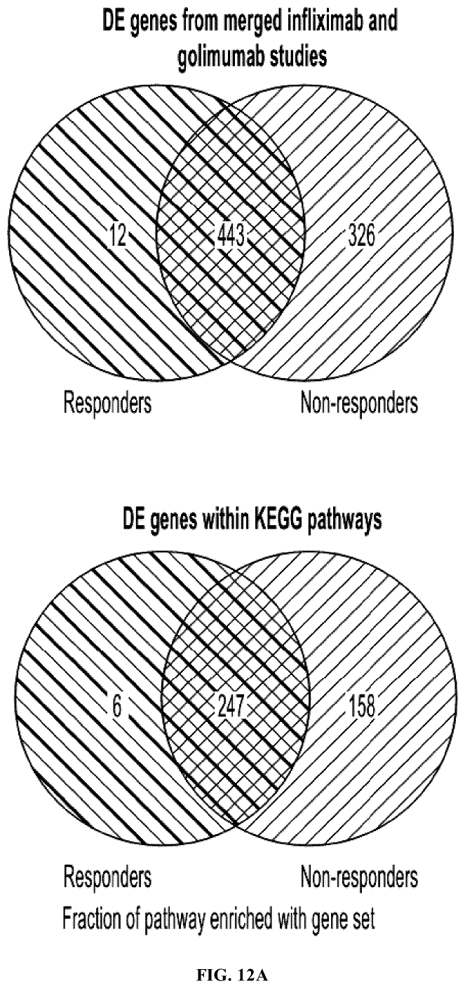

Motivated by the fact that before treatment UC active patients' gene expression profiles are not enough to distinguish responders from non-responders, methods disclosed herein may consider normal tissue controls as a comparison reference to derive more evident difference in the gene expression profiles between responders and non-responders. The following four sets of differentially expressed genes may be constructed, comparing different groups of patients and normal controls (see FIGS. 11A-11C for illustration of the sets):
1. Responders-before-after set (RBA): differentially expressed genes in responders between before- and after-treatment;
2. Non-responders-before-after set (NRBA): differentially expressed genes in non-responders between before- and after-treatment;
3. Responders set (R): differentially expressed genes between baseline responders and normal controls;
4. Non-responders set (NR): differentially expressed genes between baseline non-responders and normal controls.
   Each of these paired states are measured separately in infliximab- and golimumab-based studies.

Non-responders may not show significant changes in gene expression profiles upon treatment, thus NRBA may not contain any significantly differentially expressed genes. R, NR, and RBA sets are highly concordant and may have significant intersection size both for infliximab and golimumab studies as shown in FIG. 11B. Pairwise hypergeometric test yields p=9·10⁻⁹¹⁰ and 5·10⁻¹²⁴⁹ for the intersection between NR and R sets, p=4·10⁻⁶⁴ and 8·10⁻⁹¹ for intersection between NR and RBA sets, p=2·10⁻²²⁶ and 1·10⁻¹⁰³ for intersection of R and RBA sets in infliximab and golimumab studies, respectively.

Moreover, most RBA genes are differentially expressed in baseline responder samples relative to normal controls, indicating that treatment with a TNFi may result in reversion of the expression of a small subset of R genes. On the contrary, despite the significant fraction of RBA genes contained within the NR set, these genes are not significantly altered in non-responders after treatment with TNFi.

The RBA gene sets are almost exclusively comprised of genes contained within the R and NR sets. Moreover, as suggested by UMAP plots shown in FIGS. 8A-8B, the gene expression profiles of responders after treatment is closer to that of normal controls, while non-responders after treatment remain close to their initial pre-treatment position in the UMAP space. This suggests that to achieve low disease activity in responders, it may be sufficient for TNFi treatment to revert the expression profile of a subset of the differentially expressed genes constituting the RBA set.

### Pathway Enrichment Analysis of Differentially Expressed Genes in Responders and Non-Responders to TNFi Therapy.

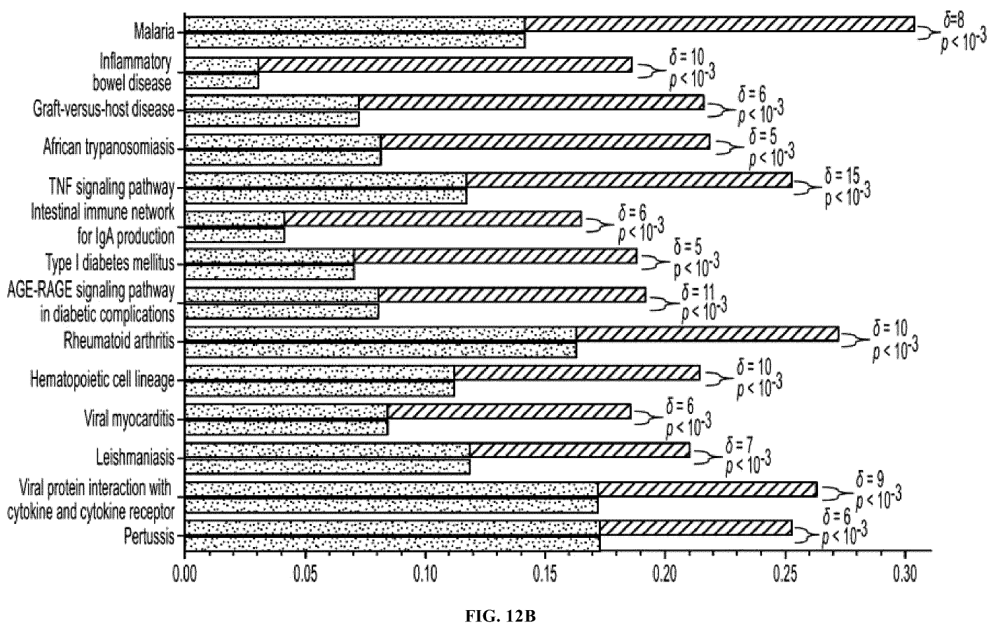

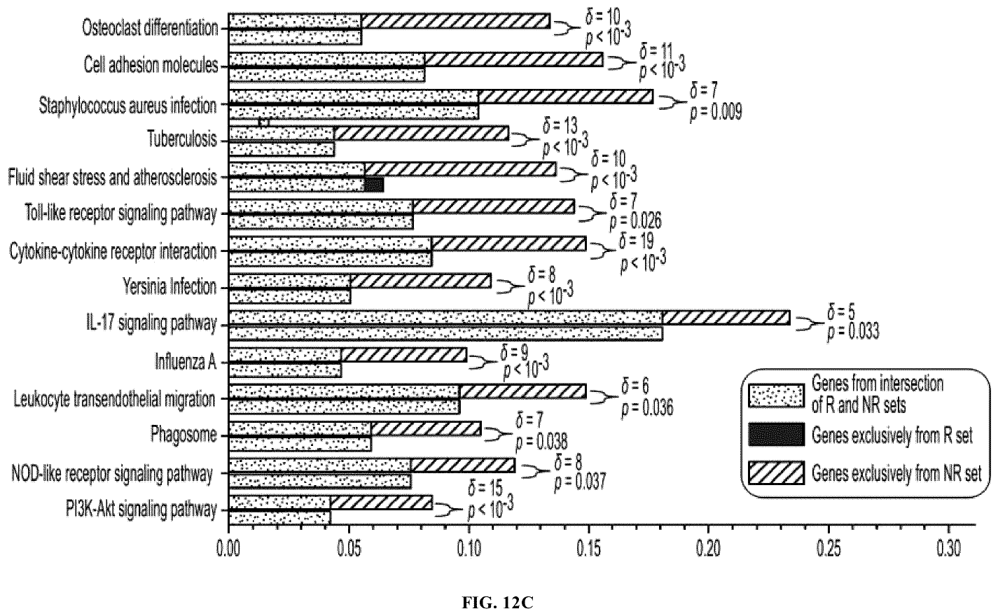

To have a better understanding of the underlying molecular mechanisms of non-response, methods disclosed herein may perform pathway enrichment analysis on the R and NR sets. For each of the KEGG pathways, the fraction of nodes that are part of the R and NR gene sets may be determined as illustrated in FIGS. 12A-12C. (See e.g., M. Kanehisa et al., “KEGG: kyoto encyclopedia of genes and genomes,” Nucleic acids research 28, 27 (2000), which is incorporated herein by reference for all purposes). Of 282 KEGG pathways that include at least one gene from the R and NR sets, 40 pathways are significantly enriched with NR genes (e.g., hypergeometric test, p<0.05). The majority of the genes in these pathways are common to the NR and R sets. To identify pathways that are more enriched in NR-exclusive genes, methods disclosed herein may perform a statistical test based on random sampling to assess the significance of difference between the number of NR-exclusive versus R-exclusive genes within the pathway. From the 40 pathways, 28 have significantly more NR-exclusive genes than R-exclusive genes are retained (p<0.05) as shown in FIGS. 12B-12C. Pathways relevant to UC such as “Inflammatory bowel disease,” “TNF signaling pathway,” “Intestinal immune network for IgA production,” “Rheumatoid arthritis,” “Cell adhesion molecules,” or “IL-17 signaling pathway” are significantly more disrupted in non-responders. This observation is supported by another pathway enrichment analysis. (See e.g., M. V. Kuleshov et al., “Enrichr: a comprehensive gene set enrichment analysis web server 2016 update,” Nucleic acids research 44, W90 (2016), which is incorporated herein by reference for all purposes). A nearly identical list of enriched biological pathways may exist between the R and NR gene sets; however, individual pathways tend to have a greater number of genes, p-value and q-values for the NR gene set. The differentially expressed genes unique to non-responders among these pathways may include genes involved in cytokine signaling (e.g., IL6, OSM, IL1A, IL1R1, IL11, CXCL8/IL8, or IL21R), receptor mediation (e.g., toll-like receptors, TLR1, TLR2, or TLR8) and signal transduction (e.g., Src-like kinases: HCK or FYN).

UC-relevant KEGG pathways are more enriched in NR-exclusive genes than that of responders as shown in FIGS. 12B-12C. This includes other inflammatory conditions such as e.g., rheumatoid arthritis and diabetes and may represent general immune system dysfunctions common to these conditions. An estimated 25-35% of patients with an autoimmune disease may develop one or more additional autoimmune disorders. (See e.g., M. Cojocaru et al., “Multiple autoimmune syndrome,” Maedica 5, 132 (2010); J.-M. Anaya et al., “The autoimmune tautology: from polyautoimmunity and familial autoimmunity to the autoimmune genes,” Autoimmune diseases 2012 (2012), which are incorporated herein by reference for all purposes). Other enriched pathways highlighted the role of the intestinal microbiome in ulcerative colitis. Genes annotated in the intestinal immune network for IgA production are enriched among non-responders. IgA antibodies are the primary secreted immunoglobulins, and pro-inflammatory bacterial taxa may be more significantly coated with IgA in inflammatory bowel disease patients than healthy controls. (See e.g., J. M. Shapiro et al., “Immunoglobulin A targets a unique subset of the microbiota in inflammatory bowel disease,” Cell Host & Microbe 29, 83 (2021), which is incorporated herein by reference for all purposes). Specifically, Staphylococcus aureus infection is one enriched bacterial KEGG pathway. Gram positive bacteria such as S. aureus induce TNF-α secretion from macrophages, and TNF-α enhances neutrophil-mediated bacterial killing. (See e.g., K. P. van Kessel et al., “Neutrophil-mediated phagocytosis of Staphylococcus aureus,” Frontiers in immunology 5, 467 (2014), which is incorporated herein by reference for all purposes). Perturbation of TNF-α affects the ability of immune system to control an S. aureus infection, leading to an elevated risk of infection after TNFi treatment. (See e.g., S. Bassetti et al., “Staphylococcus aureus in patients with rheumatoid arthritis under conventional and anti-tumor necrosis factor-alpha treatment,” The Journal of rheumatology 32, 2125 (2005), which is incorporated herein by reference for all purposes). Innate immunity plays an important role in maintaining intestinal homeostasis, as highlighted by the TLR and NOD-like signaling KEGG pathways. TLR pattern recognition receptors detect conserved structures of microbes, including those of the gut microbiota, and, upon activation, induce inflammatory signaling pathways and regulate antibody-producing B cell responses. (See e.g., L. A. O'neill et al., “The history of Toll-like receptors—redefining innate immunity,” Nature Reviews Immunology 13, 453 (2013); Z. Hua et al., “TLR signaling in B-cell development and activation,” Cellular & molecular immunology 10, 103 (2013), which are incorporated herein by reference for all purposes). TLR2, 4, 8 and 9 are upregulated in the colonic mucosa of patients with active UC relative to quiescent UC or healthy control samples. (See e.g., F Sánchez-Muñoz et al., “Transcript levels of Toll-Like Receptors 5, 8 and 9 correlate with inflammatory activity in Ulcerative Colitis,” BMC gastroenterology 11, 1 (2011), which is incorporated herein by reference for all purposes). Cytokine signaling, including the TNF-α and IL-17 pathways, are enriched among non-responders. IL-17 signaling, in addition to being a potent pro-inflammatory cytokine that amplifies TNF-α and IL-16 signaling, induces genes to recruit and activate neutrophils and promotes expression of epithelial barrier genes. (See e.g., T. Kinugasa et al., “Claudins regulate the intestinal barrier in response to immune mediators,” Gastroenterology 118, 1001 (2000); K. Maloy et al., “IL-23 and Th17 cytokines in intestinal homeostasis,” Mucosal immunology 1, 339 (2008), which are incorporated herein by reference for all purposes). Additional disruption of colonic epithelial barrier integrity in non-responders is highlighted through the enrichment of genes in the cell adhesion molecules and fluid shear stress KEGG pathways. Loss of barrier integrity increases the permeability of nutrients, water, bacterial toxins and pathogens across the epithelial barrier. (See e.g., S. C. Bischoff et al., “Intestinal permeability—a new target for disease prevention and therapy,” BMC gastroenterology 14, 1 (2014), which is incorporated herein by reference for all purposes). Overall, the pathways that are more significantly enriched suggest that UC disease biology e.g., inflammation, barrier integrity and microbiome disequilibrium, is more broadly disrupted among TNFi non-responders.

To determine if the gene expression profile of non-responders is more severely dysregulated in comparison to that of responders with respect to various pathways, methods disclosed herein may perform enrichment analysis of signaling pathways from the Kyoto® Encyclopedia of Genes and Genomes (KEGG) database. Pathways that are significantly enriched with nonresponders' differentially expressed genes are selected using the significance threshold of p_{adj.}<0.05 (hypergeometric test with Benjamini-Hochberg correction). Each selected pathway, genes that are coming exclusively from the R and NR gene sets are identified. The difference between the number of these R- and NR-exclusive genes are computed to assess its significance using the random permutation of R- and NR-exclusive labels on the remaining genes. Pathways for which there is a significant difference between the number of NR-exclusive and R-exclusive genes are retained (p_{adj.}<0.05, random permutation test with Benjamini-Hochberg correction).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of treating a subject suffering from a disease, disorder, or condition, the method comprising:
administering to a test subject a therapeutically effective amount of (i) a therapy, based at least in part on a trained machine learning classifier analyzing a disease gene expression signature to predict responsiveness of the test subject to the therapy, or (ii) a second therapy different from the therapy, based at least in part on the trained machine learning classifier analyzing the disease gene expression signature to predict non-responsiveness of the test subject to the therapy,
wherein the disease gene expression signature is determined at least in part by:
receiving gene expression data from a cohort of subjects suffering from the disease, disorder, or condition;
stratifying the cohort of subjects into two or more groups based at least in part on the gene expression data;
calculating differences in gene expression between the two or more groups of subjects and a group of non-diseased subjects;
selecting one or more genes having significant differences in gene expression between the two or more groups of subjects and the group of non-diseased subjects (“disease candidate genes”);
compiling a set of disease genes comprising the disease candidate genes; and
selecting at least a subset of the set of disease genes to thereby determine the disease gene expression signature.

2. The method of claim 1, wherein the disease gene expression signature is determined at least in part by further mapping the disease candidate genes onto a biological network, and selecting adjacent genes on the biological network having significant connection to each other or to the disease candidate genes, wherein the set of disease genes comprises the disease candidate genes and the adjacent genes.

3. The method of claim 2, wherein the biological network comprises a human interactome.

4. The method of claim 2, wherein the adjacent genes form a significant sub-network with each other or to the disease candidate genes.

5. The method of claim 2, wherein the adjacent genes are identified via a machine-learning algorithm.

6. The method of claim 5, wherein the machine-learning algorithm comprises a random walk.

7. The method of claim 1, wherein the disease, disorder, or condition comprises ulcerative colitis (UC), Crohn's disease (CD), rheumatoid arthritis (RA), juvenile arthritis, psoriatic arthritis, plaque psoriasis, ankylosing spondylitis, Guillain-Barre syndrome, Sjogren's syndrome, scleroderma, vitiligo, bipolar disorder, Graves' disease, schizophrenia, Alzheimer's disease, multiple sclerosis, Parkinson's disease, or a combination thereof.

8. The method of claim 7, wherein the disease, disorder, or condition comprises ulcerative colitis (UC).

9. The method of claim 7, wherein the disease, disorder, or condition comprises rheumatoid arthritis (RA).

10. The method of claim 7, wherein the disease, disorder, or condition comprises Alzheimer's disease.

11. The method of claim 7, wherein the disease, disorder, or condition comprises multiple sclerosis.

12. The method of claim 1, wherein the stratifying the cohort of subjects into two or more groups is random or based at least in part on whether the prior subjects do or do not respond to the therapy.

13. The method of claim 1, wherein the therapy comprises a member selected from Table 1.

14. The method of claim 1, wherein the therapy comprises an anti-TNF therapy.

15. The method of claim 1, wherein the stratifying further comprises grouping subjects from the same cohort having similar gene expression.

16. The method of claim 1, wherein the trained machine learning classifier is configured to predict responsiveness or non-responsiveness of the test subject with a negative predictive value of at least about 50%, 55%, 60%, 65%, 70%, 75%, 80%, 85%, 90%, or 95%.

17. A method of monitoring therapeutic efficacy in a subject suffering from a disease, disorder, or condition, the method comprising:
monitoring changes in a disease gene expression signature after administration of a therapy, wherein the disease gene expression signature has been determined at least in part by:
analyzing gene expression data from a cohort of subjects suffering from the same disease, disorder, or condition as the subject;
stratifying the cohort of subjects into two or more groups based on the gene expression data;
determining differences in gene expression between the two or more groups of subjects and a group of non-diseased subjects;
selecting one or more genes having significant differences in gene expression between the two or more groups of subjects and the group of non-diseased subjects (“disease candidate genes”);
compiling a set of disease genes comprising the disease candidate genes; and
selecting at least a subset of the set of disease genes to thereby determine the disease gene expression signature.

18. The method of claim 17, wherein the disease gene expression signature is determined at least in part by further mapping the disease candidate genes onto a biological network, and selecting adjacent genes on the biological network having significant connection to each other or to the disease candidate genes, wherein the set of disease genes comprises the disease candidate genes and the adjacent genes.

19. The method of claim 18, wherein the biological network comprises a human interactome.

20. The method of claim 17, wherein the adjacent genes form a significant sub-network with each other or to the disease candidate genes.

21. The method of claim 17, wherein the adjacent genes are selected by a machine-learning process.

22. The method of claim 17, wherein the disease, disorder, or condition comprises ulcerative colitis (UC), Crohn's disease (CD), rheumatoid arthritis (RA), juvenile arthritis, psoriatic arthritis, plaque psoriasis, ankylosing spondylitis, Guillain-Barre syndrome, Sjogren's syndrome, scleroderma, vitiligo, bipolar disorder, Graves' disease, schizophrenia, Alzheimer's disease, multiple sclerosis, Parkinson's disease, or a combination thereof.

23. The method of claim 17, wherein stratifying the cohort of subjects into two or more groups is random or based at least in part on whether the prior subjects do or do not respond to the therapy.

24. The method of claim 17, wherein the therapy comprises a member selected from Table 1.

25. The method of claim 17, wherein the therapy comprises an anti-TNF therapy.

26. The method of claim 17, wherein the stratifying further comprises grouping subjects from the same cohort having similar gene expression.

27. The method of claim 17, further comprising selecting a test subject for a clinical trial, based at least in part on whether the disease gene expression signature of the test subject exhibits a quantifiable change toward a disease gene expression signature of a non-diseased subject.
